(19) United States
(12) Patent Application Publication
(10) Pub. No.: US 2023/0165885 A1
(43) Pub. Date: Jun. 1, 2023

(54) TREATMENT OF EYE DISORDERS WITH URIDINE PHOSPHATE DERIVATIVES

(71) Applicant: GLIAGUARD, INC., Vineburg, CA (US)

(72) Inventor: Charles O'Neill, Sonoma, CA (US)

(21) Appl. No.: 17/914,327

(22) PCT Filed: Mar. 22, 2021
(86) PCT No.: PCT/US2021/023461

§ 371 Date: Sep. 24, 2022

Related U.S. Application Data
(60) Provisional application No. 62/993,917, filed on Mar. 24, 2020.

(51) Int. Cl. A61K 31/7072; A61K 45/06

(52) U.S. Cl. CPC A61K 31/7072; A61K 45/06

(57) ABSTRACT

The present invention relates to the treatment of eye disorders with P₂Y receptor modulating compounds, and in particular to the treatment of eye disorders such as retinal neurodegenerations, including glaucoma, age-related macular degeneration and traumatic retinal injury, posterior and anterior uveitis of the eye, and dry eye with P₂Y₆ receptor modulating compounds such as uridine phosphate derivatives and prodrugs.

### CROSS REFERENCE TO RELATED APPLICATION

This application claims priority to and the benefit of U.S. Provisional Application No. 62/993,917, filed Mar. 24, 2020, which is hereby incorporated by reference in its entirety.

### FIELD OF THE INVENTION

The present invention relates to the treatment of eye disorders with P₂Y receptor modulating compounds, and in particular to the treatment of eye disorders such as retinal neurodegenerations, including glaucoma, age-related macular degeneration and traumatic retinal injury, posterior and anterior uveitis of the eye, and dry eye with P₂Y₆ receptor modulating compounds such as uridine phosphate derivatives and prodrugs.

### BACKGROUND OF THE INVENTION

Purinergic receptors are divided into two main classes P₁ and P₂ receptors. This classification originally developed by Burnstock was based on differences in responses induced by ATP and adenosine, and the ability of methylxanthines to inhibit adenosine-mediated events but not those induced by ATP [Burnstock, 1978]. Since the initial classification of purinergic receptor, purinergic receptors are now grouped into three families P1 receptors (adenosine G-protein coupled receptors), P₂X (adenine and pyrimidine nucleotides ligand-gated ion channels) and P₂Y receptors (adenine and pyrimidine nucleotides G-protein coupled receptors).

The P₂Y receptor has been implicated in a number of disorders, including, for example, neurodegeneration, osteoporosis, immune and inflammatory responses ischemic injury, and diabetes. It has been shown that agonists of P₂Y₆ receptor counteract apoptosis induced by tumor necrosis factor alpha in astrocytoma cells[Haas 2014]. P₂Y₆ receptor was also reported to play a role in phagocytosis in microglial cells when activated by its endogenous agonist UDP. See, e.g., Malmsjo et al. BMC Pharmacol. 2003, 3, 4; Balasubrama-nian et al. Biochem. Pharmacol. 2010, 79, 1317-1332; Kim et al. Cell. Mal. Neurobiol. 2003, 23, 401-418; Mamedova et al. Pharmacol. Res. 2008, 58, 232-239; Korcok et al. J. Biol. Chem. 2005, 58, 232-239; and Koizumi et al. Nature, 2007, 446, 1091-1095.

### SUMMARY OF THE INVENTION

The present invention relates to the treatment of eye disorders with P₂Y receptor modulating compounds, and in particular to the treatment of eye disorders such as retinal neurodegenerations, including glaucoma, age-related macular degeneration and traumatic retinal injury, posterior and anterior uveitis of the eye, and dry eye with P₂Y₆ receptor modulating compounds such as uridine phosphate derivatives and prodrugs.

In another aspect of the disclosure, there is provided a method for treating retinal neurodegenerations, including glaucoma, age-related macular degeneration and traumatic retinal injury in a subject by administering a therapeutically effective amount of a P2Y receptor modulating compound.

In another aspect of the disclosure, there is provided a method for treating posterior and anterior uveitis of the eye by administering a therapeutically effective amount of a P2Y receptor modulating compound

In another aspect of the disclosure, there is provided a method for treating dry eye by administering a therapeutically effective amount of a P2Y receptor modulating compound

In some embodiments, the present invention provides methods of treating an eye disorder in a subject in need thereof comprising administering a therapeutically effective amount of a P₂Y receptor modulating compound. In some preferred embodiments, the eye disorder is selected from the group consisting of retinal neurodegenerations, including glaucoma, age-related macular degeneration and traumatic retinal injury, posterior and anterior uveitis of the eye, and dry eye disorders. In some preferred embodiments, the P₂Y receptor modulating compound is a P₂Y₆ receptor modulating compound. In some preferred embodiments, the P₂Y receptor modulating compound is a uridine diphosphate derivative or prodrug thereof. In some preferred embodiments, the uridine diphosphate derivative is a compound of formula I or a salt or a prodrug thereof or formula II of a salt or a prodrug thereof, as described in more detail below. In some preferred embodiments, the uridine diphosphate compound is a compound selected from compounds 1 to 57 as described in more detail below. In some preferred embodiments, the P₂Y receptor modulating compound is administered topically, orally, or intravenously. In some preferred embodiments, the P₂Y receptor modulating compound is administered ophthalmically. In some preferred embodiments, the P₂Y receptor modulating compound is formulated in an ophthalmically acceptable carrier. In some preferred embodiments, the ophthalmically acceptable carrier comprises one or more of an effective amount of a buffer component; an effective amount of a tonicity component; an effective amount of a preservative component; and water. In some preferred embodiments, the P₂Y receptor modulating compound is administered in combination with a second therapeutic agent. In some preferred embodiments, the second therapeutic agent is selected from the group consisting of: NMDA antagonists, antibacterials, antihistamines, decongestants, antiinflammatories, antiparasitics, miotics, sympathomimetics, anticholinergics, adrenergics, antivirals, secretagogues exemplified by agents that promote lacrimation, salivation or stimulation of release of soluble mucins and or expression of cell associated mucins that promote wettability and/or lubricity of mucosal surfaces, local anesthetics, antifungals, amoebicidals, trichomonocidals, analgesics, mydriatics, antiglaucoma drugs, carbonic anhydrase inhibitors, ophthalmic diagnostic agents, ophthalmic agents used as adjuvants in surgery, chelating agents, antineoplastics, antihypertensives, muscle relaxants, diagnostics, adrenergic anesthetics, beta blockers, alpha-2-agonists, cycloplegics, prostaglandins, derivatives thereof and mixtures thereof.

In some preferred embodiments, the present invention provides a P₂Y receptor modulating compound for use in treating an eye disorder in a subject in need thereof. In some preferred embodiments, the eye disorder is selected from the group consisting of retinal neurodegenerations, including glaucoma, age-related macular degeneration and traumatic retinal injury, posterior and anterior uveitis of the eye, and dry eye disorders. In some preferred embodiments, the P₂Y receptor modulating compound is a P₂Y₆ receptor modulating compound. In some preferred embodiments, the P2Y receptor modulating compound is a uridine diphosphate derivative or prodrug thereof. In some preferred embodiments, the uridine diphosphate derivative is a compound of formula I or a salt or a prodrug thereof or formula II of a salt or a prodrug thereof, as described in more detail below. In some preferred embodiments, the uridine diphosphate compound is a compound selected from compounds 1 to 57 as described in more detail below. In some preferred embodiments, the P₂Y receptor modulating compound is administered topically, orally, or intravenously. In some preferred embodiments, the P₂Y receptor modulating compound is administered ophthalmically. In some preferred embodiments, the P₂Y receptor modulating compound is formulated in an ophthalmically acceptable carrier. In some preferred embodiments, the ophthalmically acceptable carrier comprises one or more of an effective amount of a buffer component; an effective amount of a tonicity component; an effective amount of a preservative component; and water. In some preferred embodiments, the P₂Y receptor modulating compound is delivered via a route selected from the group consisting of topical, subconjunctival, intracameral, intravitreal, suprachoroidal, subretinal and retrobulbar routes. In some preferred embodiments, the P₂Y receptor modulating compound is formulated in a controlled release formulation. In some preferred embodiments, the controlled release formulation comprises an agent selected from the group consisting of a solid polymer formulation and a hydrogel formulation. In some preferred embodiments, the P₂Y receptor modulating compound is administered in combination with a second therapeutic agent. In some preferred embodiments, the second therapeutic agent is selected from the group consisting of: NMDA antagonists, antibacterials, antihistamines, decongestants, antiinflammatories, antiparasitics, miotics, sympathomimetics, anticholinergics, adrenergics, antivirals, secretagogues exemplified by agents that promote lacrimation, salivation or stimulation of release of soluble mucins and or expression of cell associated mucins that promote wettability and/or lubricity of mucosal surfaces, local anesthetics, antifungals, amoebicidals, trichomonocidals, analgesics, mydriatics, antiglaucoma drugs, carbonic anhydrase inhibitors, ophthalmic diagnostic agents, ophthalmic agents used as adjuvants in surgery, chelating agents, antineoplastics, antihypertensives, muscle relaxants, diagnostics, adrenergic anesthetics, beta blockers, alpha-2-agonists, cycloplegics, prostaglandins, derivatives thereof and mixtures thereof.

In some preferred embodiments, the present invention provides an ophthalmically acceptable formulation for administration to the eye comprising a therapeutically effective amount of a P₂Y receptor modulating compound and one or more of an effective amount of a buffer component; an effective amount of a tonicity component; an effective amount of a preservative component; and water. In some preferred embodiments, the effective amount a P₂Y receptor modulating compound is a dosage sufficient to treat an eye disorder selected from the group consisting of retinal neurodegenerations, including glaucoma, age-related macular degeneration and traumatic retinal injury, posterior and anterior uveitis of the eye, and dry eye disorders, when administered one or more times daily or delivered in a long acting controlled release formulation. In some preferred embodiments, the P₂Y receptor modulating compound is a P₂Y₆ receptor modulating compound. In some preferred embodiments, the P₂Y receptor modulating compound is a uridine diphosphate derivative or prodrug thereof. In some preferred embodiments, uridine diphosphate derivative is a compound of formula I or a salt or a prodrug thereof or formula II of a salt or a prodrug thereof, as described in more detail below. In some preferred embodiments, the uridine diphosphate compound is a compound selected from compounds 1 to 57 as described in more detail below. In some preferred embodiments, the formulation comprises a second therapeutic agent. In some preferred embodiments, the second therapeutic agent is selected from the group consisting of: NMDA antagonists, antibacterials, antihistamines, decongestants, antiinflammatories, antiparasitics, miotics, sympathomimetics, anticholinergics, adrenergics, antivirals, local anesthetics, secretagogues exemplified by but not limited to agents that promote lacrimation, salivation or stimulation of release of soluble mucins and or expression of cell associated mucins that promote wettability and/or lubricity of mucosal surfaces including the ocular surface), antifungals, amoebicidals, trichomonocidals, analgesics, mydriatics, antiglaucoma drugs, carbonic anhydrase inhibitors, ophthalmic diagnostic agents, ophthalmic agents used as adjuvants in surgery, chelating agents, antineoplastics, antihypertensives, muscle relaxants, diagnostics, adrenergic anesthetics, beta blockers, alpha-2-agonists, cycloplegics, prostaglandins, derivatives thereof and mixtures thereof. In some preferred embodiments, the P₂Y receptor modulating compound is a formulation selected from the group consisting of topical, subconjunctival, intracameral, intravitreal, suprachoroidal, subretinal and retrobulbar formulations. In some preferred embodiments, the P₂Y receptor modulating compound is formulated in a controlled release formulation. In some preferred embodiments, the controlled release formulation comprises an agent selected from the group consisting of a solid polymer formulation and a hydrogel formulation.

### DEFINITIONS

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Administering” or “administration of a substance, a compound or an agent to a subject can be carried out using one of a variety of methods known to those skilled in the art. For example, a compound or an agent can be administered, intravenously, arterially, intradermally, intra-muscularly, intraperitonealy, intravenously, subcutaneously, sublingually, orally (by ingestion), intranasally (by inhalation), intraspinally, intracerebrally, and transdermally (by absorption, e.g., through a skin duct). Specific ocular administration routes include topical administration to the ocular surface (cornea and/or conjunctiva), retrobulbar administration, intracameral administration, intravitreal administration, suprachoroidal administration and sub-retinal administration. A compound or agent can also appropriately be introduced by rechargeable or biodegradable polymeric devices or other devices, e.g., patches and pumps, or formulations, which provide for the extended, slow or controlled release of the compound or agent. The polymeric materials may be solid implantable materials or may be designed such as to maintain prolonged contact with the ocular surface (a commercial example is lacriserts, see world wide web at bausch.com/ecp/our-products/rx-pharmaceuticals/rx-pharmaceuticals/lacrisert) or be formed into punctal plugs that slowly release the test article (e.g., see https://ois.net/punctal-plugs-for-sustained-delivery/) or O rings that are placed into the conjunctival fomices (e.g. https: .aao.org/eye-health/news/new-glaucoma-treatment-ring-shows-promise). Other embodiments as to therapeutic constructs are exemplified but not limited to materials applied topically or injected into and/or around the eye that form hydrogels whose polymerization is triggered by changes in temperature, pH or ionic composition. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject’s size, health and age, the nature and extent of cognitive impairment or other symptoms of the condition being treated, such as dry eye or other eye disorder. The skilled worker can readily determine the effective amount for a given situation by routine experimentation.

“Treating” a condition or patient refers to taking steps to obtain beneficial or desired results, including clinical results. Beneficial or desired clinical results include, but are not limited to, alleviation, amelioration, or slowing the progression, of one or more symptoms associated with a neuronal disorder, including neurodegeneration and traumatic brain injury, as well as pain. In certain embodiments, treatment may be prophylactic. Exemplary beneficial clinical results are described herein.

“Prodrug” or “pharmaceutically acceptable prodrug” refers to a compound that is metabolized, for example hydrolyzed or oxidized, in the host after administration to form the compound of the present disclosure (e.g., compounds of formula I or II). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. The prodrugs of this disclosure are metabolized to produce a compound which are agonists of the P₂Y₆ receptors.

As used herein, the term chemical formula includes information about the spatial arrangement of bonds in a chemical but not necessarily the exact isomer; while the term molecular formula refers to the number of atoms of each element in the compound.

“Alkyl” refers to a monovalent straight-chain, branched or cyclic saturated aliphatic hydrocarbon radical. Preferably, the alkyl group is a straight chain radical having 1 to 40 carbon atoms. More preferably, it is an alkyl radical of from 5 to 31 carbon atoms, most preferably 13 to 17 carbon atoms. Typical alkyl radicals include pentyl, hexyl, tridecanyl, tetradecanyl, nonadecanyl, docosanyl, triacontanyl, hentriacontanyl and the like. Preferably this term denotes an acyclic carbon or a saturated acyclic carbon chain represented by the formula CnH2n+1 wherein n is an integer of from 1 to 31.

“Alkenyl” refers to a monovalent, straight-chain, branched or cyclic, unsaturated aliphatic hydrocarbon radical having one or more, preferably one, double bond. Preferably, the alkenyl radical has from 2 to 40 carbon atoms. More preferably, it is an alkenyl radical of from 6 to 30 carbon atoms, most preferably 14 to 22 carbon atoms. Typical alkenyl groups include hexenyl, tridecenyl, tetradecenyl, nonadecenyl, docosenyl, triacontenyl, hentriacontenyl and the like. Preferably this term denotes an acyclic carbon chain which contains a carbon-to-carbon double bond and is represented by the formula CnH2n-1 wherein n is an integer of from 2 to 40.

“Alkylene” refers to a divalent, straight-chain, branched or cyclic, saturated aliphatic hydrocarbon radical. Preferably, the alkylene group has from 1 to 12 carbon atoms. This term denotes an acyclic carbon or a saturated acyclic carbon chain represented by the formula CnH2n-2 wherein n is an integer of from 1 to 12. More preferably, it is a lower alkylene of from 1 to 7 carbon atoms, most preferably from 1 to 4 carbon atoms, e.g., methylene.

The term “aliphatic” as used herein means a straight chained or branched alkyl, alkenyl or alkynyl. It is understood that alkenyl or alkynyl embodiments need at least two carbon atoms in the aliphatic chain. Aliphatic groups typically contains from 1 (or 2) to 12 carbons, such as from 1 (or 2) to 4 carbons.

The term “aryl” as used herein means a monocyclic or bicyclic carbocyclic aromatic ring system. Phenyl is an example of a monocyclic aromatic ring system. Bicyclic aromatic ring systems include systems wherein both rings are aromatic, e.g., naphthyl, and systems wherein only one of the two rings is aromatic, e.g., tetralin.

The term “heterocyclic” as used herein means a monocyclic or bicyclic non-aromatic ring system having 1 to 3 heteroatom or heteroatom groups in each ring selected from O, N, NH, S, SO, or SO2 in a chemically stable arrangement. In a bicyclic non-aromatic ring system embodiment of “heterocyclyl”, one or both rings may contain said heteroatom or heteroatom groups. In another heterocyclic ring system embodiment, a non-aromatic heterocyclic ring may optionally be fused to an aromatic carbocycle.

Examples of heterocyclic rings include 3-1H-benzimidazol-2-one, 3-(1-alkyl)-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolane, benzodithiane, and 1,3-dihydro-imidazol-2-one.

The term “heteroaryl” as used herein means a monocyclic or bicyclic aromatic ring system having 1 to 3 heteroatom or heteroatom groups in each ring selected from O, N, NH or S in a chemically stable arrangement. In such a bicyclic aromatic ring system embodiment of “heteroaryl” both rings may be aromatic; and one or both rings may contain said heteroatom or heteroatom groups.

Examples of heteroaryl rings include 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

The term “cycloalkyl or cycloalkenyl” refers to a monocyclic or fused or bridged bicyclic carbocyclic ring system that is not aromatic. Cycloalkenyl rings have one or more units of unsaturation. Exemplary cycloalkyl or cycloalkenyl groups include cyclopropyl, cyclobutyl, cyclopentyl, cyclohexyl, cyclohexenyl, cycloheptyl, cycloheptenyl, norbomyl, adamantyl and decalinyl.

As used herein, the carbon atom designations may have the indicated integer and any intervening integer. For example, the number of carbon atoms in a (C1-C4)-alkyl group is 1, 2, 3, or 4. It should be understood that these designation refer to the total number of atoms in the appropriate group. For example, in a (C3-C10)-heterocyclyl the total number of carbon atoms and heteroatoms is 3 (as in aziridine), 4, 5, 6 (as in morpholine), 7, 8, 9, or 10.

“Pharmaceutically acceptable salt” or “salt” is used herein to refer to an agent or a compound according to the disclosure that is a therapeutically active, non-toxic base and acid salt form of the compounds. The acid addition salt form of a compound that occurs in its free form as a base can be obtained by treating said free base form with an appropriate acid such as an inorganic acid, for example, a hydrohalic such as hydrochloric or hydrobromic, sulfuric, nitric, phosphoric and the like; or an organic acid, such as, for example, acetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclic, salicylic, p-aminosalicylic, pamoic and the like. See, e.g., WO 01/062726.

### DETAILED DESCRIPTION

The present invention relates to the treatment of eye disorders with P₂Y receptor modulating compounds, and in particular to the treatment of eye disorders such as retinal neurodegenerations, including glaucoma, age-related macular degeneration and traumatic retinal injury, posterior and anterior uveitis of the eye, and dry eye with P₂Y₆ receptor modulating compounds such as uridine phosphate derivatives and prodrugs.

P₂Y₁ receptors have been identified in the cornea, ciliary processes, and trabecular meshwork and the retina. P₂Y₂, P₂Y₄, and P₂Y₆ receptors are expressed in the cornea and ciliary processes, neural retina and retinal pigment epithelium. The retinal pigmented epithelium also expressed the P₂Y₁₁ receptors. P₂Y_{11,} and P₂Y₁₃ receptors are present on lacrimal Glands. [Sanderson 2014, Pintor 2004, Ohtomo 2011]

P₂Y receptors have been shown to multiple actions in the eye: modulating tear production, corneal wound healing, intraocular pressure, and retinal physiology. Nucleotides can affect both the volume and composition of tears. Nucleotides cause an increase in the content of secreted mucins, lysozyme, and other tear proteins. [Sanderson 2014] In the lens, ATP activated P₂Y receptors have been shown to help maintain lens transparency by activating a Na/K ATPase pump in the lens epithelium. UTP applied topically to the rabbit cornea stimulate a MAPK-dependent increase in epithelial cell migration to accelerate wound healing.[Pintor 2004] P₂Y_{R} agonists, including those active at of P₂Y and P₂Y receptors lower IOP [Markovskaya 2008]. P2Y1 receptors reduces ischemia-induced apoptosis of cells in the retina. [Pannicke 2014].

There is a need for new ligands, such as agonists, active at P₂Y and especially for P₂Y₆ receptors that are useful in therapeutic preparations for the treatment of ocular disorders responsive to the activation of these receptor. In the eye, these disorders include retinal neurodegenerations, traumatic eye injury, posterior and anterior uveitis and dry eye. These compounds are typically selective ligands of the P₂Y₆ receptor; however, given the complexity of receptor expression in the eye and the overlapping responses and selectivity of P₂Y receptors that activation of other P₂Y receptors will mediate the response. In certain embodiments, the compounds as described herein are agonists, which activate the P₂Y receptor. In certain embodiments, the compounds as described herein are prodrugs of agonists, which activate the P₂Y receptor.

Accordingly, in some preferred embodiments, the present invention provides for the use of modulators of the P₂Y receptors (e.g., P₂Y₆ receptors) for treatment of eye disorders including but not limited to retinal neurodegenerations, traumatic eye injury, posterior and anterior uveitis and dry eye. Suitable modulating compounds and their synthesis are described in U.S. Pat. Publ. 2019/0309009, which is incorporated herein by reference in its entirety. In particular, suitable modulating compounds include but are not limited to uridine diphosphate (UDP) compounds, prodrugs and derivatives.

In some embodiments, the P₂Y receptor modulating compounds for use in treating eye disorders are a compound of formula I:

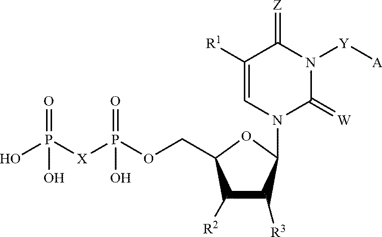

or a prodrug or salt thereof, wherein:
A is a 3- to 10-membered aromatic or non-aromatic ring having up to 5 heteroatoms independently selected from N, O, S, SO, or SO₂, wherein the aromatic or non-aromatic ring is independently and optionally substituted with one or more R⁷;
X is independently selected from —O—,—S—,—N(R⁵)— and a (C1-C3)-aliphatic group independently and optionally substituted with one or more R⁴;
Y is a bond or a (C1-C5)-aliphatic group independently and optionally substituted with one or more R⁴;
Z and W are each independently selected from ═O, ═S. ═N(R⁵), and ═NOR⁵;

R¹ is selected from:
—H, halogen. —OR⁵, —CN, —CF₃, —OCF₃ and a (C1-C6)-aliphatic group optionally substituted with one or more R⁷;
R² and R³ are each independently selected from —OR⁵, —SR⁵, —NR⁵R⁶, —OC(O)R⁵, —OC(O)NR⁵R⁶, and —OC(O)OR⁵; preferably, R² and R³ are each independently selected from —OR⁵, —SR⁵, —NR⁵R⁶ and —OC(O)R⁵;
each occurrence of R⁴ is independently selected from: a halogen. —OR⁵, —NO₂, —CN, — CF₃, —OCF₃, —R⁵, 1,2-methylenedioxy, 1,2-ethylenedioxy, —N(R⁵)₂, —SR⁵, —SOR⁵, —SO₂R⁵, —SO₂N(R⁵)₂, —SO₃R⁵, —C(O)R⁵, —C(O)C(O)R⁵, —C(O)CH₂C(O)R⁵, —C(S)R⁵, —C(S)OR⁵, —C(O)OR⁵, —C(O)C(O)OR⁵, —C(O)C(O)N(R⁵)₂, —OC(O)R⁵, —C(O)N(R⁵)₂, —OC(O)N(R⁵)₂, —C(S)N(R⁵)₂, —(CH₂)_{0—2}NHC(O)R⁵, —N(R⁵)N(R⁵)COR⁵, — N(R⁵)N(R⁵)C(O)OR⁵, —N(R⁵)N(R⁵)CON(R⁵)₂, —N(R⁵)SO₂R⁵, —N(R⁵)SO₂N(R⁵)₂, —N(R⁵)C(O)OR⁵, —N(R⁵)C(O)R⁵, —N(R⁵)C(S)R⁵, —N(R⁵)C(O)N(R⁵)₂, —N(R⁵)C(S)N(R⁵)₂, —N(COR⁵)COR⁵,—N(OR⁵)R⁵,—C(═NH)N(R⁵)₂, —C(O)N(OR⁵)R⁵, —C(═NOR⁵)R⁵, —OP(O)(OR⁵)₂, —P(O)(R⁵)₂, —P(O)(OR⁵)₂, or —P(O)(H)(OR⁵);
each occurrence of R⁵ is independently selected from: H—, (C1-C12)-aliphatic-, (C3-C10)-cycloalkyl- or -cycloalkenyl-. [(C3-C10)-cycloalkyl or -cycloalkenyl]-(C1-C12>aliphatic-, (C6-C10)-aryl-, (C6-C10)-aryl-(C1-C12)aliphatic-, (C3-C10)-heterocyclyl-, (C6-C10)-heterocyclyl-(C1-C12)aliphatic-, (C5-C10)-heteroaryl-, and (C5-C10)-heteroaryl-(C1-C12)-aliphatic-; wherein two R⁵ groups bound to the same atom optionally form a 3- to 10-membered aromatic or non-aromatic ring having up to 3 heteroatoms independently selected from N, O, S, SO, or SO₂, wherein said ring is optionally fused to a (C6-C10)aryl, (C5-C10)heteroaryl, (C3-C10)cycloalkyl, or a (C3-C10)heterocyclyl; and wherein each R⁵ group is independently and optionally substituted with one or more R⁷;
R⁶ is selected from: R⁵, —C(O)R⁵, —C(O)OR⁵, —C(O)N(R⁵)₂ and —S(O)₂R⁵;
each occurrence of R⁷ is independently selected from: halogen. —OR⁸, —NO₂, —CN, —CF₃, —OCF₃, —R⁸, oxo, thioxo, 1,2-methylenedioxy, 1,2-ethylenedioxy, —N(R⁸)₂, —SR⁸, —SOR^{B}, —SO₂R⁸, —SO₂N(R⁸)₂, —SO₃R⁸, —C(O)R⁸, —C(O)C(O)R⁸, —C(O)CH₂C(O)R⁸, — C(S)R⁸, —C(S)OR⁸, —C(O)OR⁸, —C(O)C(O)OR⁸, —C(O)C(O)N(R⁸)₂, —OC(O)R⁸, — C(O)N(R⁸)₂, —OC(O)N(R⁸)₂, —C(S)N(R⁸)₂, —(CH₂)_{0—2}NHC(O)R⁸, —N(R⁸)N(R⁸)COR⁸, —N(R⁸)N(R⁸)C(O)OR⁸, —N(R⁸)N(R⁸)CON(R^{S})₂, —N(R⁸)SO₂R⁸, —N(R⁸)SO₂N(R⁸)₂, — N(R⁸)C(O)OR⁸, —N(R⁸)C(O)R⁸, —N(R⁸)C(S)R⁸, —N(R⁸)C(O)N(R⁸)₂, —N(R⁸)C(S)N(R⁸)₂, —N(COR⁸)COR⁸, —N(OR⁸)R⁸, —C(═NH)N(R⁸)₂, —C(O)N(OR⁸)R⁸, —C(═NOR⁸)R⁸, —OP(O)(OR⁸)₂, —P(O)(R⁸)₂,—P(O)(OR⁸)₂, or —P(O)(H)(OR⁸);
each occurrence of R⁸ is independently selected from: H— and (C1-C6)-aliphatic-.

In some embodiments, the salt is a pharmaceutically acceptable salt of a compound of formula I, such as a sodium salt.

In certain embodiments of compound of formula I, A is a (C5-C10)-aromatic ring having up to 5 heteroatoms independently selected from N, O and S, wherein the aromatic ring is independently and optionally substituted with one or more R⁷. In some embodiments, A is an optionally substituted 5- or 6-membered aromatic ring having up to 2 heteroatoms selected from N, O and S. In some embodiments, A is an optionally substituted bi-cyclic aromatic ring having up to 4 heteroatoms selected from N, O and S. For example, A is an aromatic group selected from:

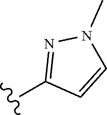

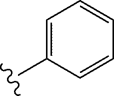

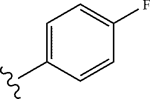

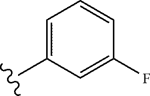

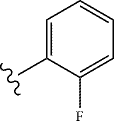

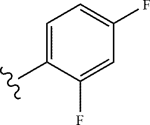

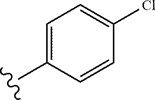

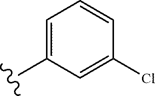

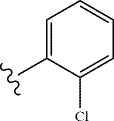

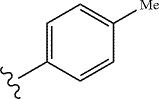

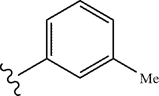

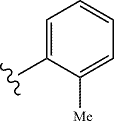

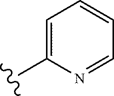

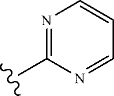

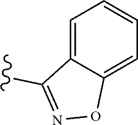

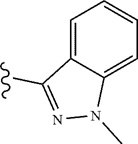

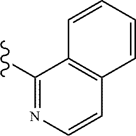

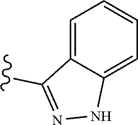

wherein A is optionally further substituted with one or more R⁷.

In certain embodiments, A is selected from:

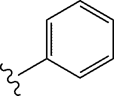

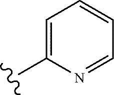

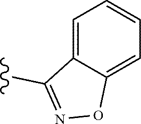

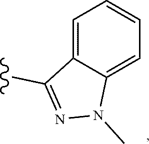

wherein A is optionally further substituted with one or more R⁷.

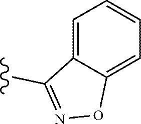

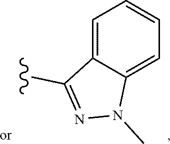

In some embodiments, A is optionally further substituted with one or more R⁷.

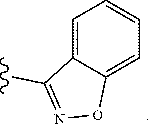

In another embodiment, A is optionally substituted with one or more R⁷. In some of the above embodiments of A, each occurrence of R⁷ is independently selected from halogen, —CF₃, —OCF₃, —C1—C4 aliphatic (e.g., —C1—C4 alkyl), and —O(C1-C4 aliphatic) (e.g., —O(C1-C4 alkyl)).

In certain embodiments, the present disclosure provides compounds of formula I, where X is —O—.

In some embodiments, the present disclosure also provides compounds of formula I, where R¹ is —H, bromine, iodine, methyl, ethyl or —CF₃. In some embodiments, R¹ is —H.

According to certain embodiments, the present disclosure provides a compound of formula I, where Z is ═O or ═S. In some embodiments, Z is ═O.

In some embodiments, the compound of the present disclosure has a W that is ═O or ═S. In some embodiments, W is ═O.

According to certain embodiments, the present disclosure provides a compound of formula I, where Y is a C1-aliphatic group optionally substituted with one or more R⁴. For example. Y is —CH₂—. In some embodiments. Y is a C2-aliphatic group optionally substituted with one or more R⁴. In some embodiments, Y is —CH₂—C(R⁴)₂—, such as —CH₂—CH₂—. In another embodiment, Y is —CH₂—C(R⁴)₂—, where each R⁴ is independently selected from halogen. In some embodiments, Y is —CH₂—C(R⁴)₂—, where both occurrences of R⁴ are —F. In another embodiment, Y is —CH₂—C(R⁴)₂—, where each occurrence of R⁴ is independently a (C1-C3)-aliphatic group. In yet another embodiment, Y is —CH₂—C(R⁴)₂— , where both occurrences of R⁴ are —CH₃.

In some embodiments, the present disclosure provides a compound of formula I, where R² and R³ are each independently —OR⁵. In some embodiments. R² is —OH. In another embodiment, R³ is —OH.

The disclosure also includes various combinations of A, X, Y, Z, W, R² and R³ as described above. These combinations can in turn be combined with any or all of the values of the other variables described above. For example, in some embodiments. Y is a C1— or C2-aliphatic group optionally substituted with one or more R⁴ and X is —O—. In another embodiment, Y is a C1— or C2-aliphatic group optionally substituted with one or more R⁴; X is —O—; and Z is ═O. In another embodiment, Y is a C1— or C2-aliphatic group optionally substituted with one or more R⁴; X is —O—; Z is ═O; and W is ═O. In yet another embodiment. Y is a C1-or C2-aliphatic group optionally substituted with one or more R⁴; X is —O—; Z is ═O; W is ═O; and R¹ is selected from —H, bromine, iodine, methyl, ethyl, and —CF₃, for example, R¹ is —H. In a further embodiment. Y is a C1— or C2-aliphatic group optionally substituted with one or more R⁴; X is —O—; Z is ═O; W is ═O; and R¹ is selected from —H, bromine, iodine, methyl, ethyl, and —CF₃; and A is selected from the following groups:

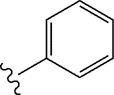

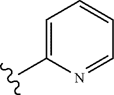

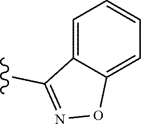

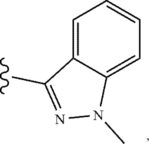

wherein A is optionally further substituted with one or more R⁷, for example. A is optionally substituted

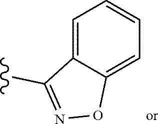

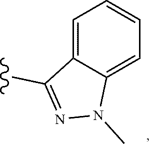

In a further embodiment, Y is a C1 — or C2-aliphatic group optionally substituted with one or more R⁴; X is —O—; Z is ═O; W is ═O; and R¹ is selected from —H, bromine, iodine, methyl, ethyl, and —CF₃; A is selected from the following group:

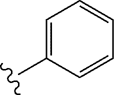

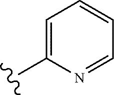

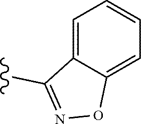

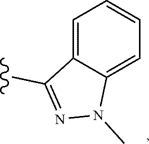

wherein A is optionally further substituted with one or more R⁷;
and R² and R³ are each independently —OR⁵, for example, R² and R³ are each independently —OH. In some of the above embodiments, each occurrence of R⁷ is independently selected from halogen, —CF₃, —OCF₃, —C1—C4 aliphatic (e.g., —C1—C4 alkyl), and —O(C1-C4 aliphatic) (e.g., —O(C 1-C4 alkyl)).

The present disclosure also provides a compound of formula II for use in treating eye disorders:

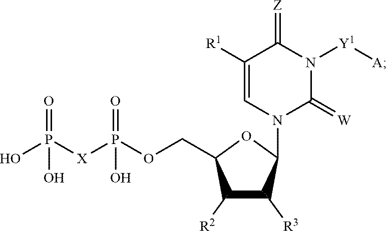

or a prodrug or salt thereof, wherein:
A is selected from: a phenyl group that is substituted with at least one (C1 -C5)-aliphatic group or halogen: a naphthalene group; a 5- to 10-membered heteroaryl group having up to 5 heteroatoms independently selected from N, O and S; and a 3- to 10-membered non-aromatic ring having up to 5 heteroatoms independently selected from N, O, S. SO, or SO₂; wherein A is optionally further substituted with one or more R⁴;
X is independently selected from —O—, —S—, —N(R⁵)— and a (C1-C3)-aliphatic: group independently and optionally substituted with one or more R⁴;
Y¹ is a (C1-C5)-aliphatic group substituted with at least one oxo and further independently and optionally substituted with one or more R⁴;
Z and W are each independently selected from ═O, ═S. ═N(R⁵), and ═NOR⁵;
R¹ is selected from: —H, halogen, —OR⁵, —CN, —CF₃, —OCF₃ and a (C1-C6)-aliphatic-group optionally substituted with one or more R⁴;
R² and R³ are each independently selected from —OR⁵, —SR⁵, —NR⁵R⁶, —OC(O)R⁵, — OC(O)NR⁵R⁶, and —OC(O)OR⁵; preferably, R² and R³ are each independently selected from —OR⁵, —SR⁵,—NR⁵R⁶ and —OC(O)R⁵;
each occurrence of R⁴ is independently selected from: halogen, —OR⁵, —NO², —CN, —CF₃, —OCF₃, —R⁵, oxo, thioxo, 1,2-methylenedioxy, 1,2-ethylenedioxy, —N(R⁵)₂, —SR⁵, — SOR⁵, —SO₂R⁵, —SO₂NCR⁵)₂, —SO₃R⁵, —C(O)R⁵, —C(O)C(O)R⁵, —C(O)CH₂C(O)R⁵, — C(S)R⁵, —C(S)OR⁵, —C(O)OR⁵, —C(O)C(O)OR⁵, —C(O)C(O)N(R⁵)₂, —OC(O)R⁵, — C(O)N(R⁵)₂, —OC(O)N(R⁵)₂, —C(S)N(R⁵)₂, —(CH₂)_{0—2}NHC(O)R⁵, —N(R⁵)N—(R⁵)COR⁵, — N(R⁵)N(R⁵)C(O)OR⁵, —N(R⁵)N(R⁵)CON(R⁵)₂, —N(R⁵)SO₂R⁵, —N(R⁵)SO₂N(R⁵)₂, — N(R⁵)C(O)OR⁵, —N(R⁵)C(O)R⁵, —N(R⁵)C(S)R⁵, —N(R⁵)C(O)N(R⁵)₂, —N(R⁵)C(S)N(R⁵)₂, —N(COR⁵)COR⁵, —N(OR⁵)R⁵, —C(═NH)N(R⁵)₂, —C(O)N(OR⁵)R⁵, —C(═NOR⁵)R⁵, — OP(O)(OR⁵)₂, —P(O)(R⁵)₂, —P(O)(OR⁵)₂, or —P(O)(H)(OR⁵);
each occurrence of R⁵ is independently selected from: H—, (C1-C12)-aliphatic-, (C3-C10)-cycloalkyl- or -cycloalkenyl-, [(C3-C10)-cycloalkyl or -cycloalkenyl]-(C1-C12)-aliphatic-, (C6-C10)-aryl-, (C6-10)-aryl-(C1-C 12)aliphatic-. (C3-C10)-heterocyclyl-, (C6-C 10)-heterocyclyl-(C1-C12)aliphatic-. (C5-C10)-heteroaryl-, and (C5-C10)-heteroaryl-(C1-C12)-aliphatic-; wherein two R⁵ groups bound to the same atom optionally form a 3- to 10-membered aromatic or non-aromatic ring having up to 3 heteroatoms independently selected from N, O, S. SO, or SO₂, wherein said ring is optionally fused to a (C6-C10)aryl, (C5-C10)heteroaryl, (C3-C10)cycloalkyl, or a (C3-C10)heterocyclyl; and wherein each R⁵ group is independently and optionally substituted with one or more R⁷;
R⁶ is selected from: R⁵, —C(O)R⁵, —C(O)OR⁵, —C(O)N(R⁵)₂ and —S(O)₂R⁵;
each occurrence of R⁷ is independently selected from: halogen, —OR⁸, —NO₂, —CN, — CF₃, —OCF₃, —R⁸, oxo, thioxo, 1,2-methylenedioxy, 1,2-ethylenedioxy, —N(R⁸)₂, —SR⁸, —SOR^{B}, — SO₂R⁸, —SO₂N(R⁸)₂, —SO₃R⁸, —C(O)R⁸, —C(O)C(O)R⁸, —C(O)CH₂C(O)R⁸, —C(S)R⁸, —C(S)OR⁸, —C(O)OR⁸, —C(O)C(O)OR⁸, —C(O)C(O)N(R⁸)₂, —OC(O)R⁸, — C(O)N(R⁸)₂, —OC(O)N(R⁸)₂, —C(S)N(R⁸)2, —(CH₂)_{0—2}NHC(O)R⁸, —N(R⁸)N(R⁸)COR⁸, — N(R⁸)N(R⁸)C(O)OR⁸, —N(R⁸)N(R⁸)CON(R⁸)₂, —N(R⁸)SO₂R⁸, —N(R⁸)SO₂N(R⁸)₂, — N(R⁸)C(O)OR⁸, —N(R⁸)C(O)R⁸, —N(R⁸)C(S)R⁸, —N(R⁸)C(O)N(R⁸)₂, —N(R⁸)C(S)N(R⁸)₂, —N(COR⁸)COR⁸, —N(OR⁸)R⁸, —C(═NH)N(R⁸)₂, —C(O)N(OR⁸)R⁸, —C(═NOR⁸)R⁸, — OP(O)(OR⁸)₂, —P(O)(R⁸)₂, —P(O)(OR⁸)₂, or —P(O)(H)(OR⁸);
each occurrence of R⁸ is independently selected from: H— and (C1-C6)-aliphatic-.

In some embodiments, the salt is a pharmaceutically acceptable salt of a compound of formula II, such as a sodium salt.

In certain embodiments of compound of formula II, A is selected from the following groups:

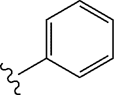

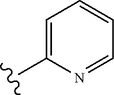

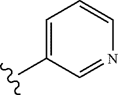

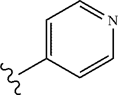

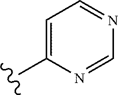

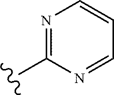

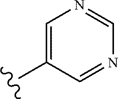

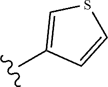

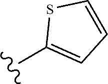

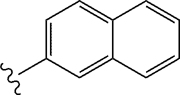

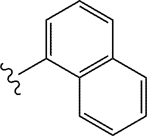

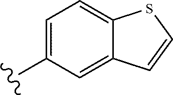

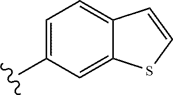

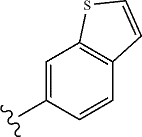

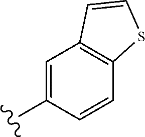

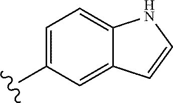

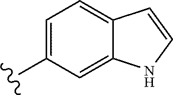

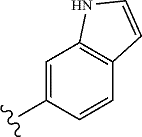

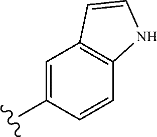

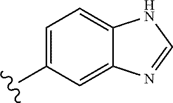

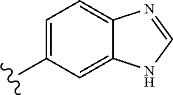

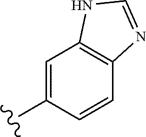

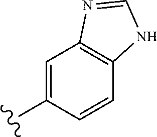

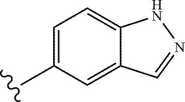

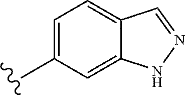

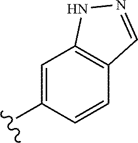

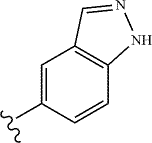

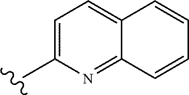

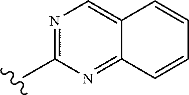

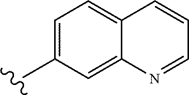

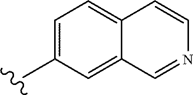

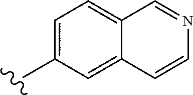

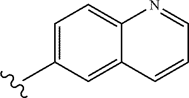

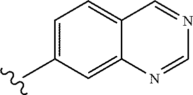

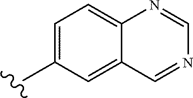

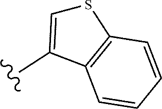

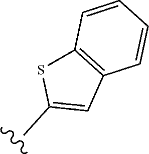

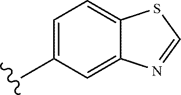

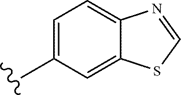

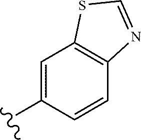

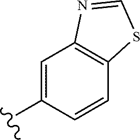

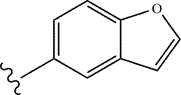

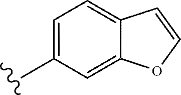

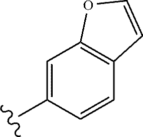

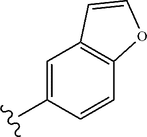

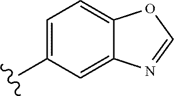

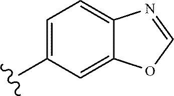

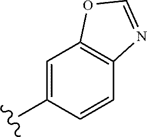

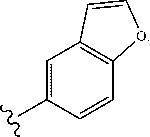

where A is optionally substituted with one or more R⁴

In other embodiments of compound of formula II, A is selected from the following groups:

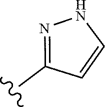

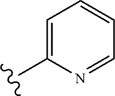

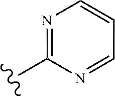

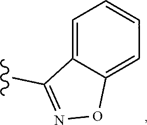

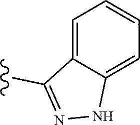

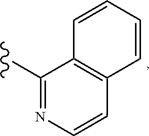

where A is optionally substituted with one or more R⁴.

In such embodiments. A is one of the following groups:

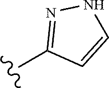

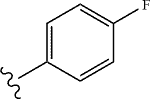

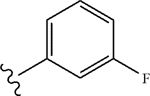

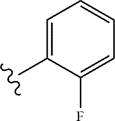

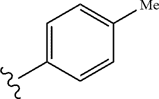

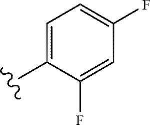

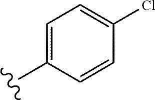

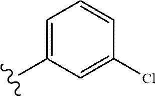

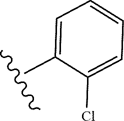

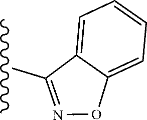

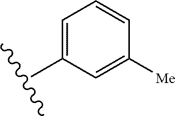

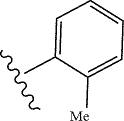

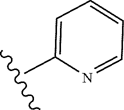

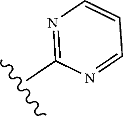

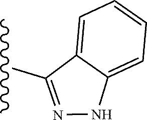

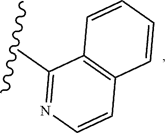

where A is optionally further substituted with one or more R⁴.

In some embodiments, A is selected from:

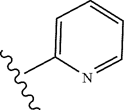

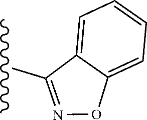

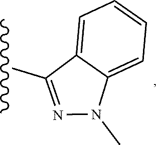

where A is optionally further substituted with one or more R⁴.

In some embodiments, A is

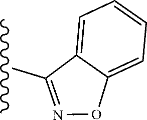

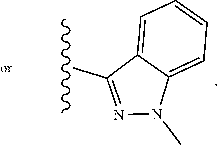

where A is optionally further substituted with one or more R⁴

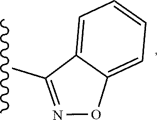

In a further embodiment, A is optionally substituted with one or more R⁴. In some of the above embodiments of A, each occurrence of R⁴ is independently selected from halogen.— CF₃, —OCF₃, —C1—C4 aliphatic (e.g., —C1—C4 alkyl), and —O(C1-C4 aliphatic) (e.g.,— O(C1-C4 alkyl)).

In some embodiments, Y¹ is a C2-aliphatic group substituted with at least one oxo and optionally further substituted with one or more R⁴, and A is selected from: a phenyl group that is substituted with at least one (C1-C5)-alipliatic group or halogen; a naphthalene group; and a 6-membered monocyclic or a 9- to 10-membered bicyclic heteroaryl group having up to 5 heteroatoms independently selected from N. O and S. wherein the bicyclic heteroaryl group has a 6-membered aryl or heteroaryl ring that is directly connected to Y¹;
wherein A is optionally further substituted with one or more R⁴. In some such embodiments, Y¹ is a C2-aliphatic group substituted with one oxo, and A is selected from:

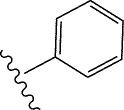

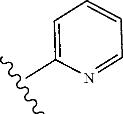

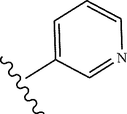

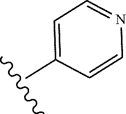

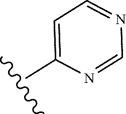

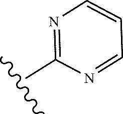

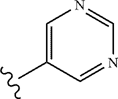

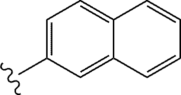

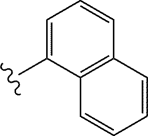

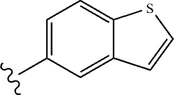

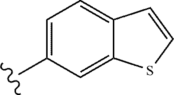

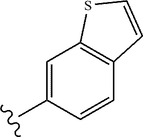

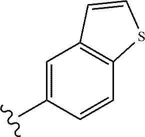

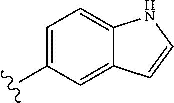

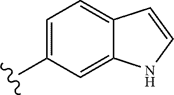

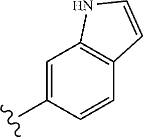

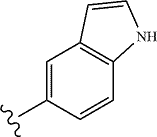

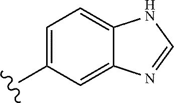

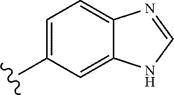

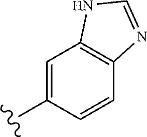

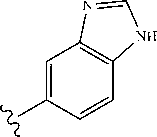

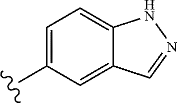

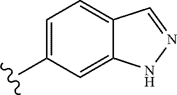

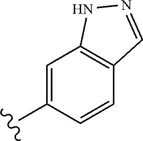

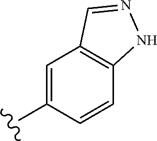

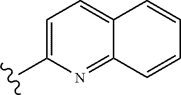

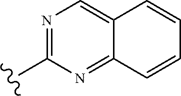

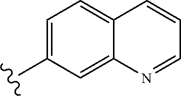

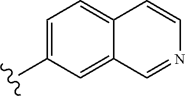

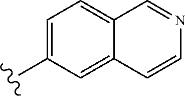

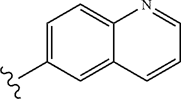

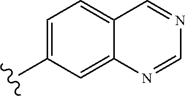

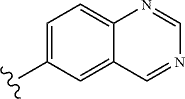

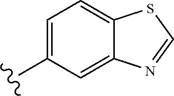

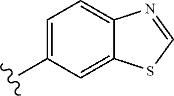

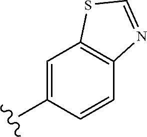

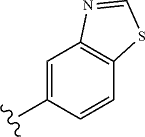

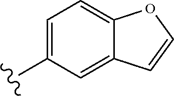

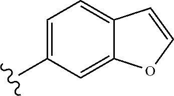

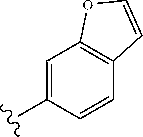

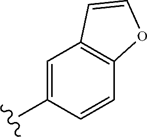

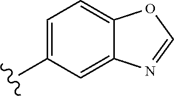

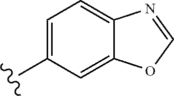

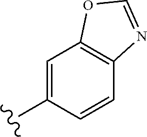

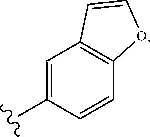

wherein A is optionally further substituted with one or more R⁴.

According to certain embodiments, the present disclosure provides a compound of formula II, where X is —O—.

In some embodiments of the compound of formula II, R¹ is —H, bromine, iodine, methyl, ethyl or —CF₃. In some embodiments. R¹ is —H.

According to certain embodiments, the present disclosure also provides a compound of formula II, where Z is ═O or ═S. In some embodiments, Z is ═O

In some embodiments of the compound of formula II, W is ═O or ═S. In some embodiments, W is ═O.

According to certain embodiments, the present disclosure also provides a compound of formula II, where Y¹ is a C1-aliphatic group substituted with oxo. In some embodiments, Y¹ is a C2-aliphatic group substituted with at least one oxo and optionally further substituted with one or more R⁴. In another embodiment, Y¹ is —C(O)—C(R⁴)₂— or —C(R⁴)₂—C(O)—, for example, —C(O)—CH₂— or —CH₂—C(O)—. In a further embodiment. Y¹ is —C(O)— C(R⁴)₂— or —C(R⁴)₂—C(O)—, where each R⁴ is independently selected from halogen. For example, Y¹ is —C(O)—C(R ⁴)₂— or—C(R⁴)₂—C(O)—, where both occurrences of R⁴ in are —F. In yet another embodiment, Y¹ is —C(O)—C(R⁴)₂ —or —C(R⁴)₂—C(O)—, where each R⁴ is independently a (C1-C3)-aliphatic group. For example, Y¹ is —C(O)—C(R⁴)₂ —or— C(R⁴)₂—C(O)—, where both occurrences of R⁴ are —CH₃.

In some embodiments of compound of formula II, R² and R³ are each independently —OR⁵. In some embodiments, R² is —OH In another embodiment. R³ is —OH.

The disclosure also includes various combinations of A. X. Y¹, Z, W, R′, R² and R³ as described above. These combinations can in turn be combined with any or all of the values of the other variables described above. For example, in some embodiments, Y¹ is a C1-aliphatic group substituted with an oxo or a C2-aliphatic group substituted with at least one oxo and optionally further substituted with one or more R⁴ and X is —O—. In another embodiment, Y¹ is a C1-aliphatic group substituted with an oxo or a C2-aliphatic group substituted with at least one oxo and optionally further substituted with one or more R⁴; X is —O—; and Z is ═O In another embodiment, Y¹ is a C 1-aliphatic group substituted with an oxo or a C2-aliphatic group substituted with at least one oxo and optionally further substituted with one or more R⁴; X is —O—; Z is ═O; and W is ═O. In yet another embodiment. Y¹ is a C1-aliphatic group substituted with an oxo or a C2-aliphatic group substituted with at least one oxo and optionally further substituted with one or more R⁴; X is —O—; Z is =O; W is =O; and R¹ is selected from —H, bromine, iodine, methyl, ethyl, and —CF₃, for example. R¹ is —H. In a further embodiment. Y¹ is a C1-aliphatic group substituted with an oxo or a C2-aliphatic group substituted with at least one oxo and optionally further substituted with one or more R⁴; X is —O—; Z is ═O; W is ═O; and R¹ is selected from —H, bromine, iodine, methyl, ethyl, and —CF₃; and A is selected from the following groups:

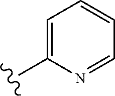

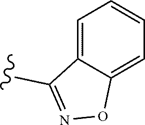

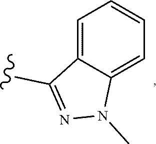

wherein A is optionally further substituted with one or more R⁴, for example, A is optionally further substituted

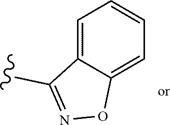

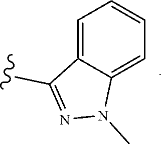

In yet a further embodiment. Y¹ is a C1-aliphatic group substituted with an oxo or a C2-aliphatic group substituted with at least one oxo and optionally further substituted with one or more R⁴; X is —O—; Z is ═O; W is ═O; and R¹ is selected from —H, bromine, iodine, methyl, ethyl, and —CF₃; A is selected from the following group:

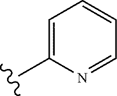

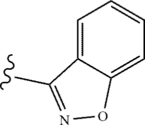

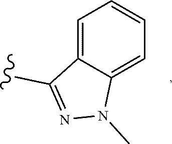

wherein A is optionally further substituted with one or more R⁴; and R² and R³ are each independently —OR⁵, for example. R² and R³ are each independently —OH. In some of the above embodiments, each occurrence of R⁷ is independently selected from halogen. -CF_{3,} —OCF₃, —C1—C4 aliphatic (e.g., —C1—C4 alkyl), and —O(C1-C4 aliphatic) (e.g., —O(C1-C4 alkyl)).

Examples of particular compounds of the present invention for use in treating eye disorders include but are not limited to:

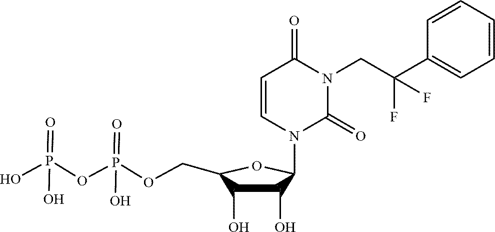

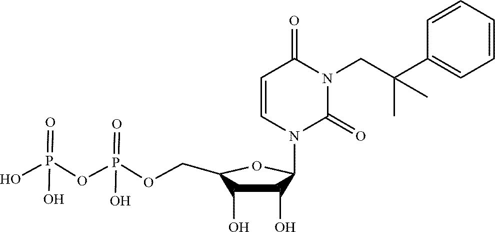

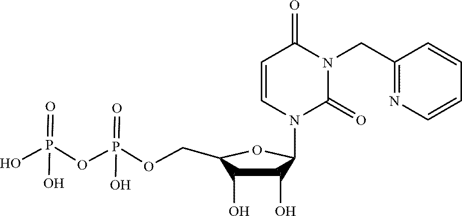

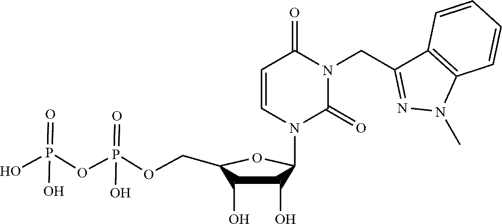

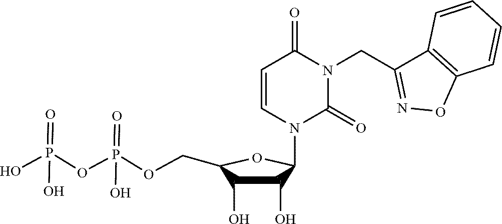

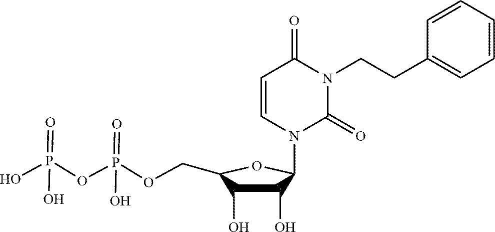

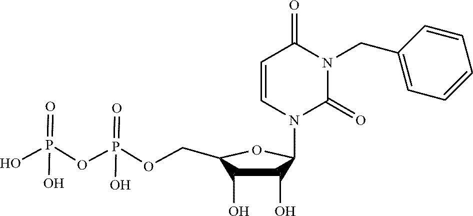

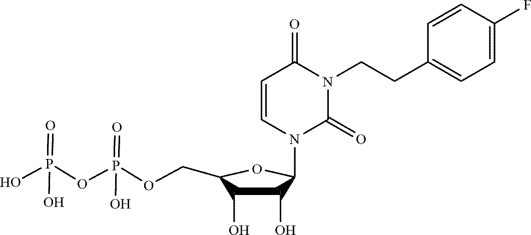

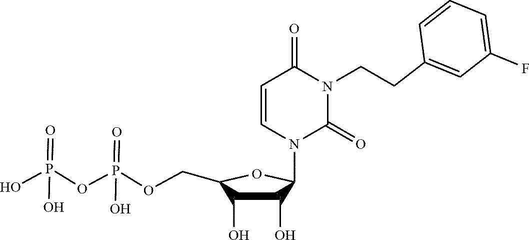

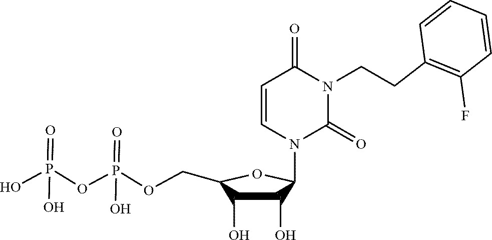

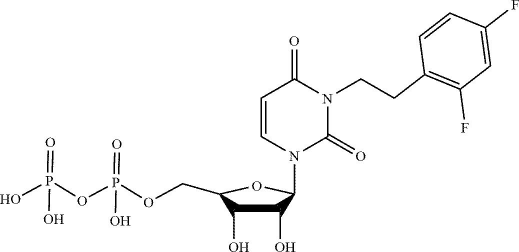

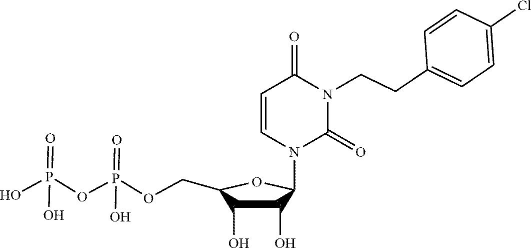

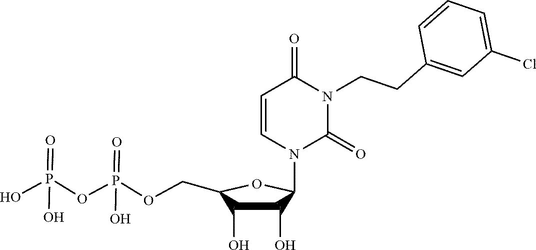

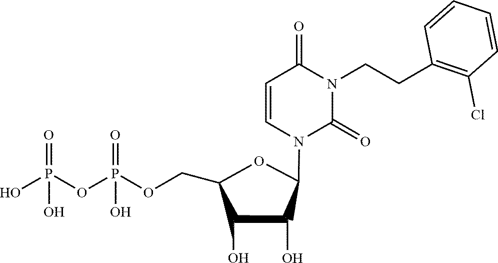

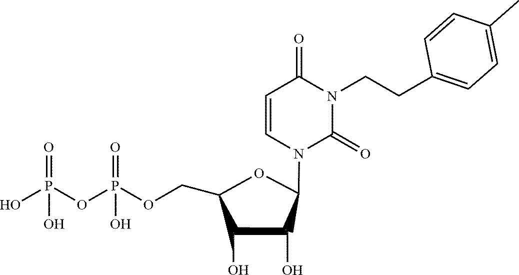

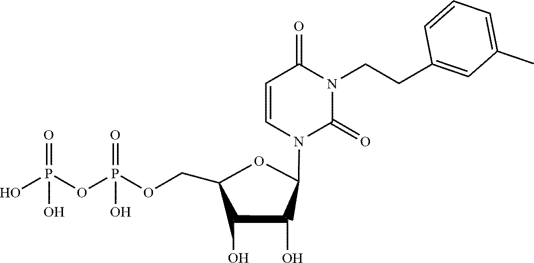

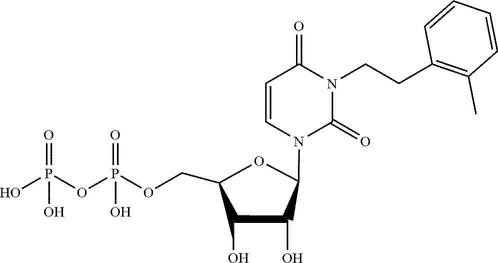

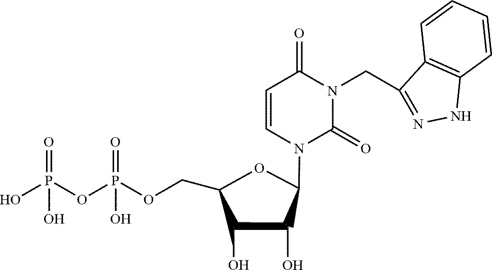

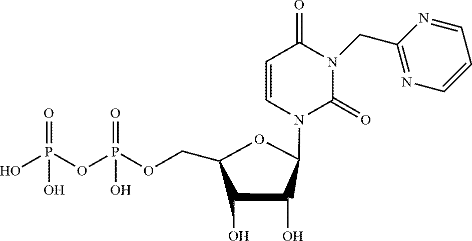

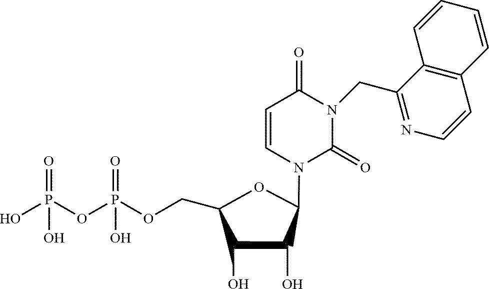

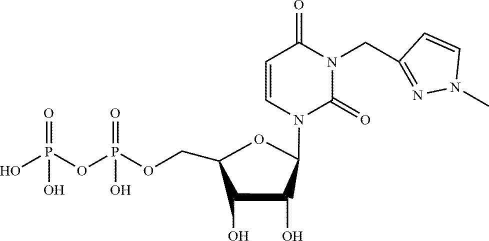

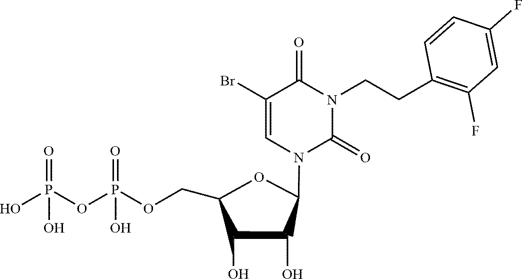

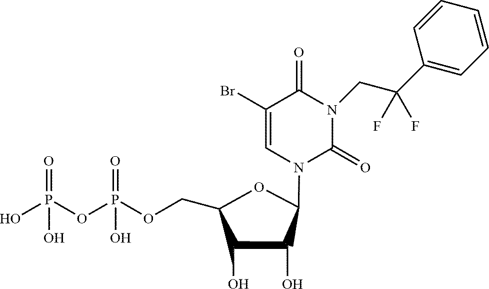

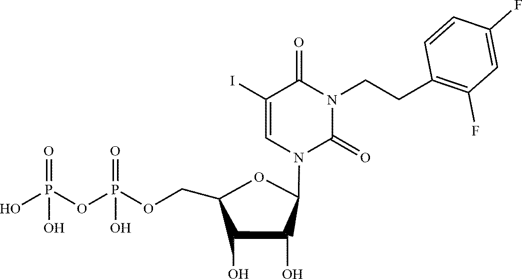

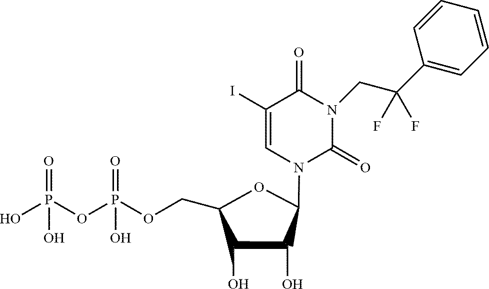

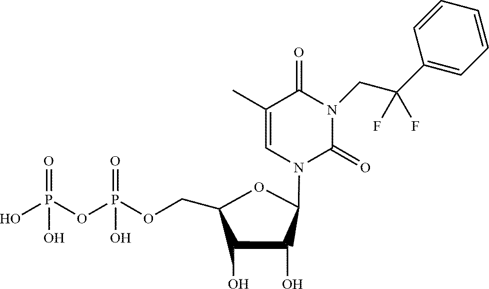

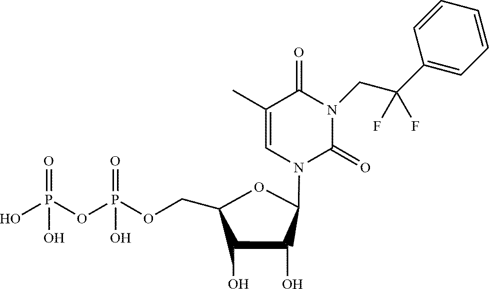

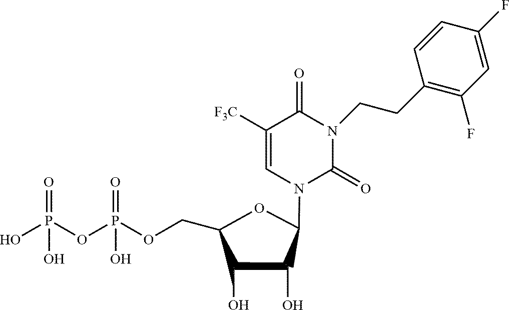

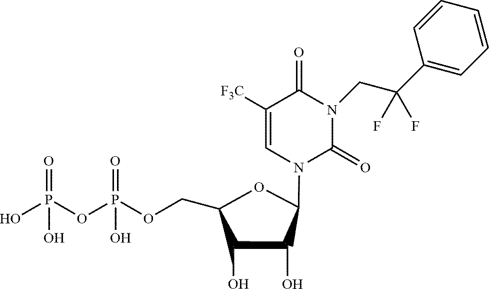

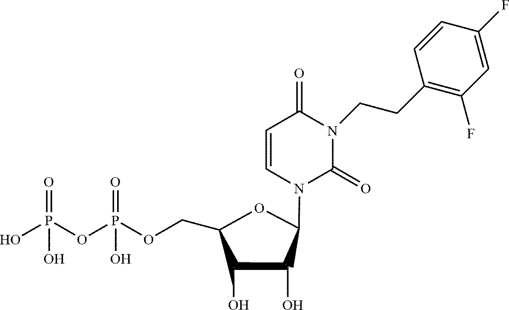

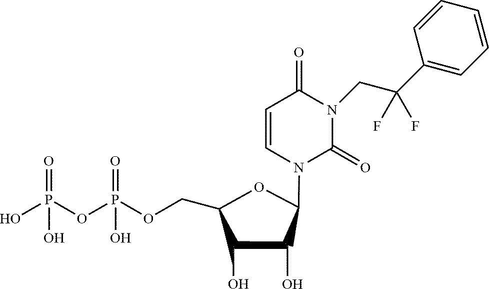

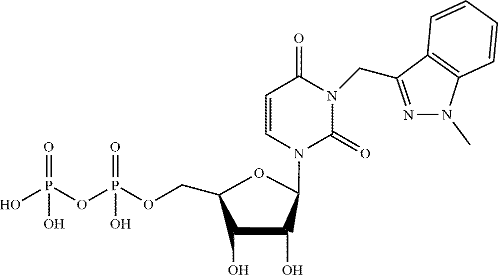

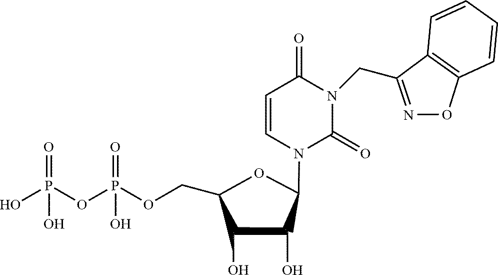

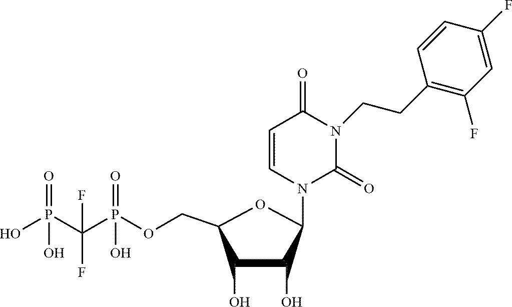

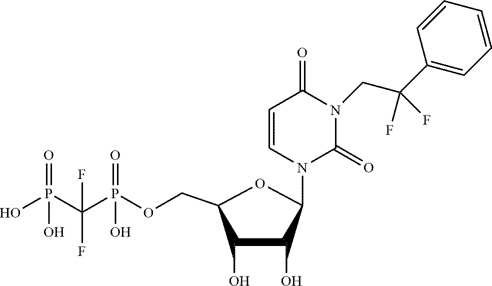

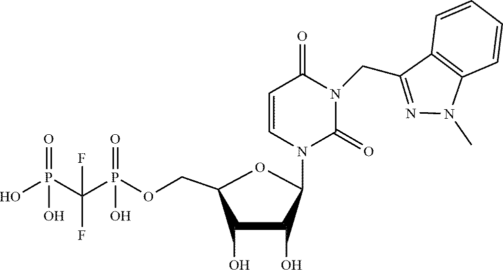

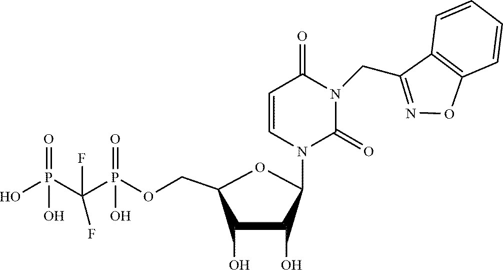

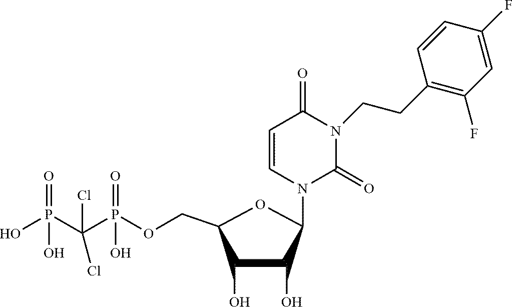

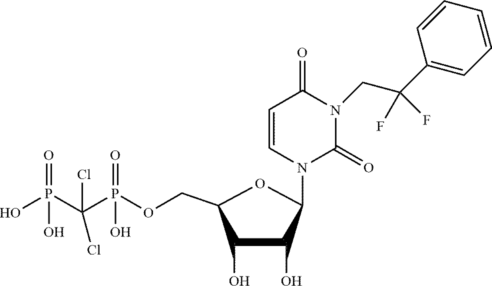

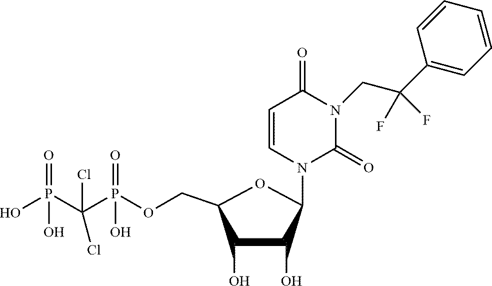

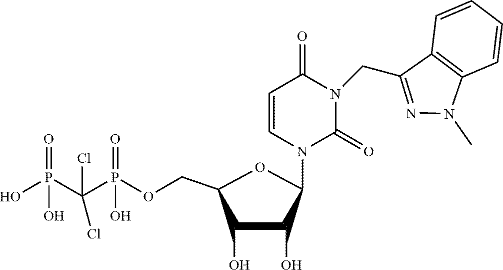

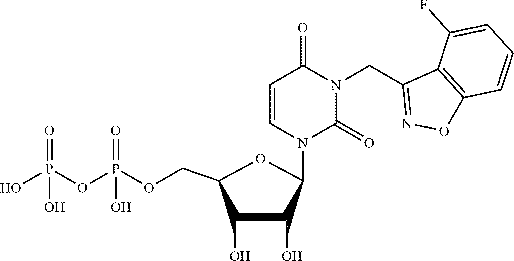

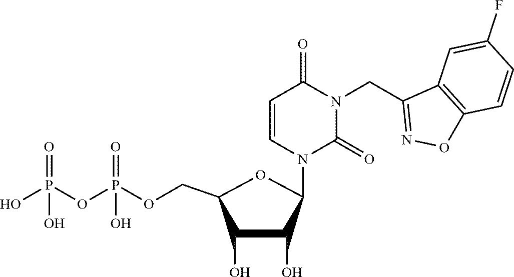

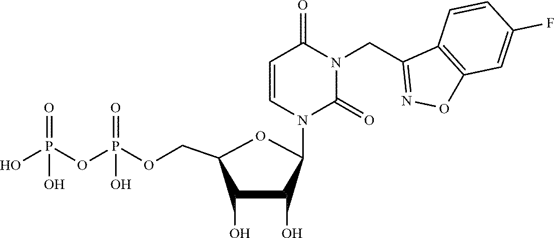

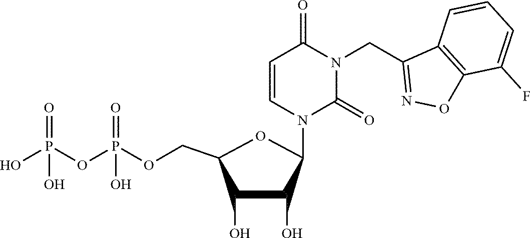

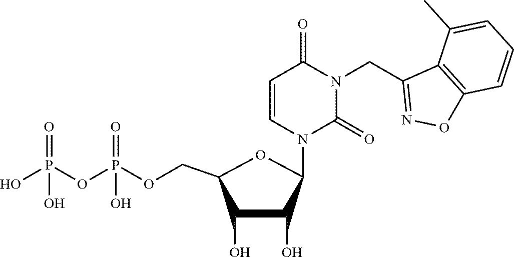

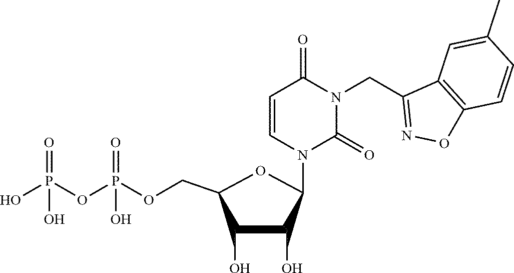

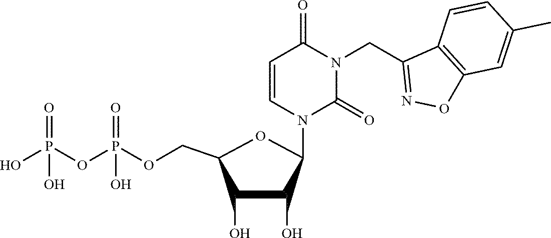

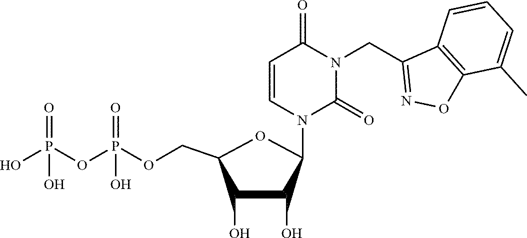

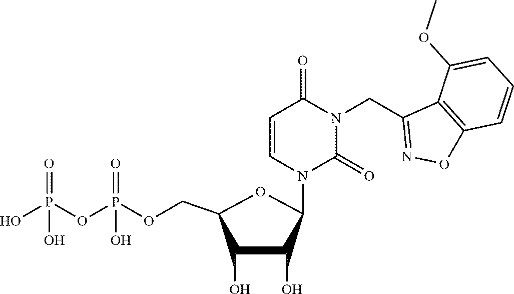

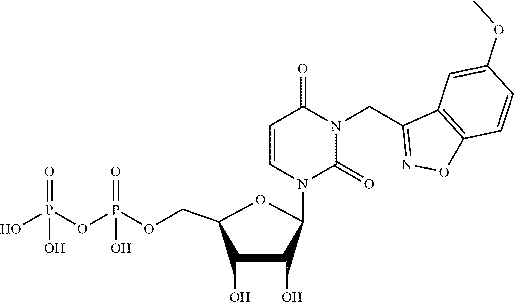

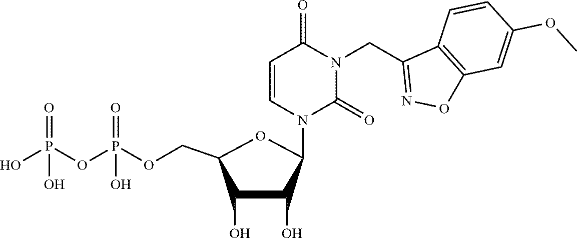

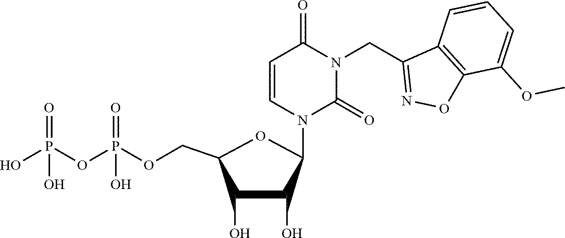

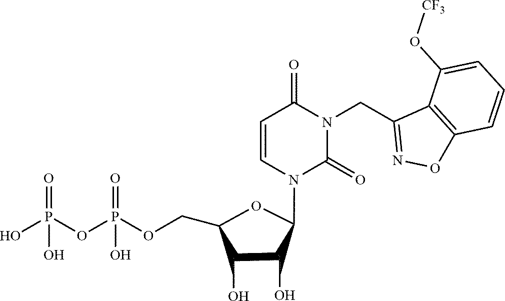

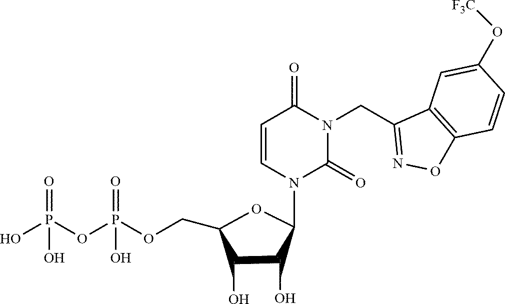

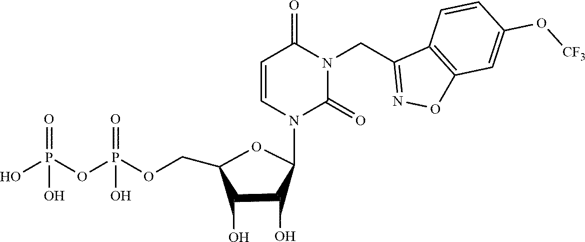

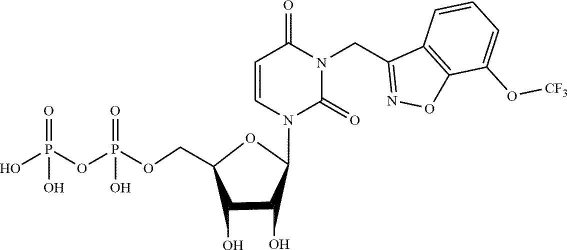

or pharmaceutically acceptable salts thereof. In certain embodiments, the pharmaceutically acceptable salt is a sodium salt.

In another embodiment, the present disclosure provides a pharmaceutical composition comprising a pharmaceutically acceptable carrier and a compound of formula I or II or pharmaceutically acceptable salt form thereof for use in treating a disorder of the eye.

The P₂Y receptor modulating compounds of this disclosure may be prepared in general by methods known to those skilled in the art. Scheme 1 below illustrates a general synthetic route to the compounds of the present disclosure. Other equivalent schemes, which will be readily apparent to the ordinary skilled organic chemist, may alternatively be used to synthesize various portions of the molecules as illustrated by the general scheme below.

Scheme 1

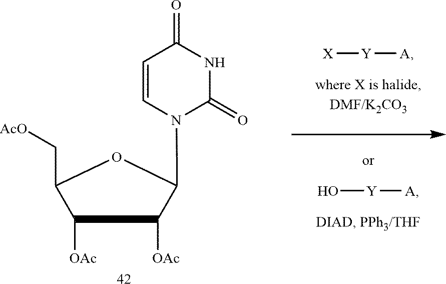

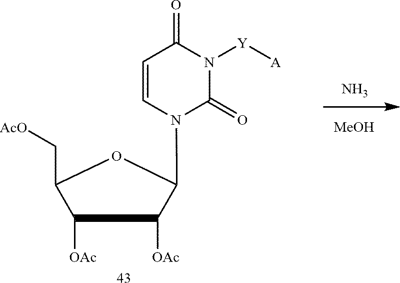

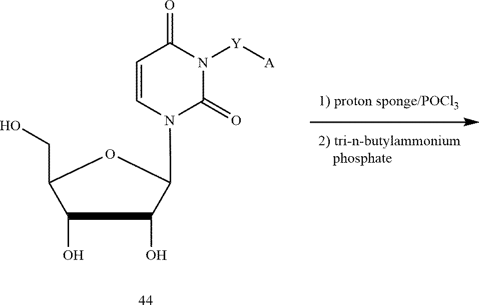

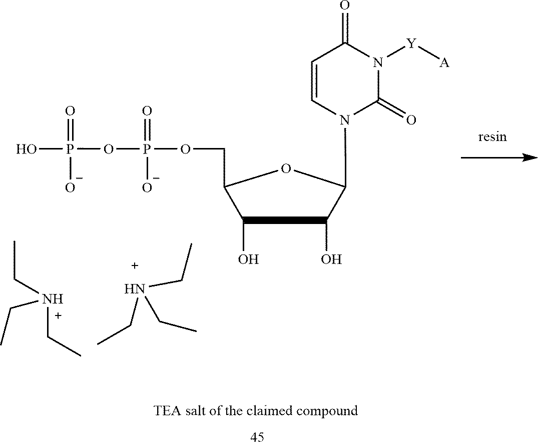

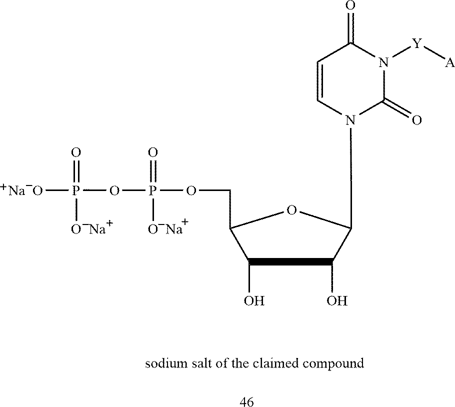

The present disclosure provides a prodrug of a compound of formula I or II or pharmaceutically acceptable salt form thereof. In some embodiments, the prodrug of the instant application includes biologically labile or cleavable protecting groups at one or both phosphate groups of a compound of formula 1 or II, e.g., moieties that are cleaved or hydrolyzed in the patient’s body to generate the compound of formula I or II or a salt thereof. In some embodiments, the prodrugs of the present disclosure can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the compound of formula I or II

In certain embodiments, the prodrug includes two biologically labile or cleavable protecting groups on the terminal phosphate group of a compound of formula I or II. In other embodiments, the prodrug includes three biologically labile or cleavable protecting groups on both phosphate groups of a compound of formula I or II.

In certain embodiments, the prodrug of the present disclosure for use in treating eye disorders has the formula:

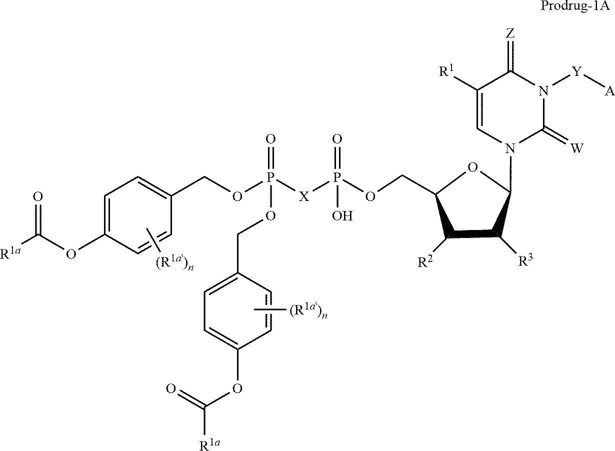

or a salt thereof, wherein:
A, X, Y, Z, W, R¹, R² and R³ are as defined above in formula I;
each n is independently 0-4;
each occurrence of R^{1a} is a group independently selected from aliphatic (such as —(C1—C6)-alkyl), heterocyclyl, cycloalkyl, cycloalkenyl, aryl and heteroaryl, wherein said aliphatic, heterocyclyl, cycloalkyl, cycloalkenyl, aryl or heteroaryl is unsubstituted or substituted with at least one R⁷ as defined above in formula I; and each occurrence of R^{1a′} is independently selected from —H and R⁷ as defined above in formula I.

In some embodiments of prodrug-IA, at least one R^{1a} is an alkyl group, such as methyl, ethyl, isopropyl or t-butyl. In some embodiments of prodrug-IA, at least one R^{1a} is an optionally substituted phenyl. In preferred embodiments, n is 0. In certain embodiments of prodrug-IA, both occurrences of R^{1a} are the same.

In certain embodiments, the prodrug of the present disclosure for use in treating eye disorders has the formula:

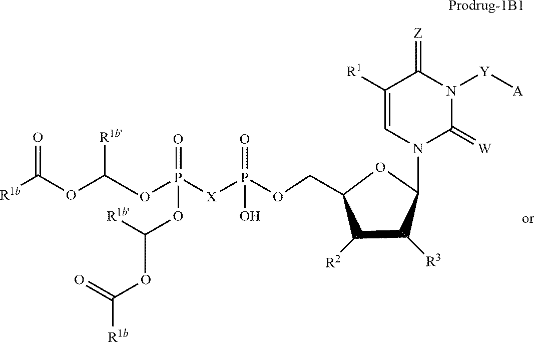

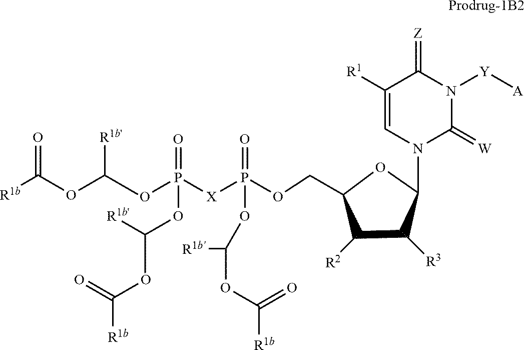

or a salt thereof, wherein: A, X, Y, Z, W, R′, R² and R³ are as defined above in formula I; each occurrence of R^{1b} is a group independently selected from aliphatic (such as —(C1—C6)-alkyl), heterocyclyl, cycloalkyl, cycloalkenyl, aryl and heteroaryl, wherein said aliphatic, heterocyclyl, cycloalkyl, cycloalkenyl, aryl or heteroaryl is unsubstituted or substituted with at least one R⁷ as defined above in formula I; and each occurrence of R^{1b′} is independently —H, —(C1—C6)-aliphatic (such as —(C1—C6)-alkyl) or —(C3—C6)-cycloalkyl; preferably, each occurrence of R^{1b′} is independently —H or —(C1—C6)-aliphatic (such as —(C1—C6)-alkyl).

In some embodiments of prodrug-IB1 or prodrug-IB2, at least one occurrence of R^{1b} is an alkyl group, such as methyl, ethyl, isopropyl or t-butyl. In some embodiments of prodrug-IB1 or prodrug-IB2, at least one occurrence of R^{1b′} is —H. In certain embodiments of prodrug-IB1 or prodrug-IB2, at least one occurrence of R^{1b′} is a —(C1—C6)-alkyl group, such as methyl, ethyl or isopropyl. In some embodiments of prodrug-IB1 or prodrug-IB2, all the occurrences of R^{1b} are the same. In some embodiments, all the occurrences of R^{1b′} are the same.

In certain embodiments, the prodrug of the present disclosure for use in treating eye disorders has the formula:

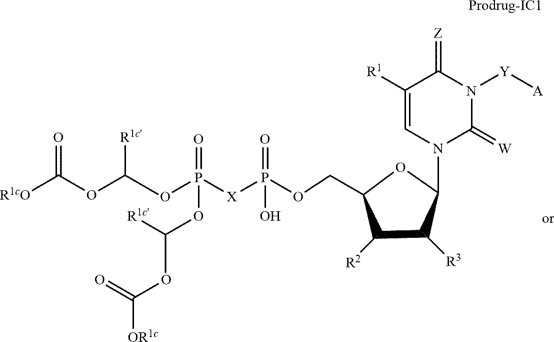

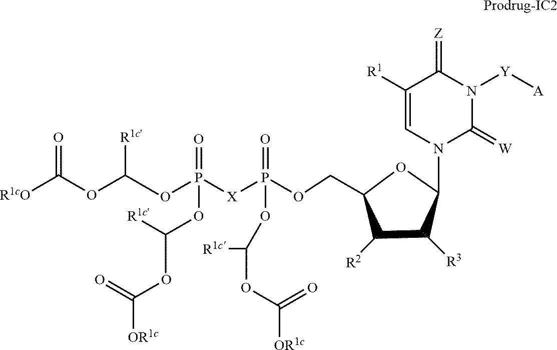

or a salt thereof, wherein A, X, Y, Z, W, R¹, R² and R³ are as defined above in formula I; each occurrence of R^{1c} is a group independently selected from aliphatic (such as —(C1—C6)-alkyl), heterocyclyl, cycloalkyl, cycloalkenyl, aryl and heteroaryl, wherein said aliphatic, heterocyclyl, cycloalkyl, cycloalkenyl, aryl or heteroaryl is unsubstituted or substituted with at least one R⁷ as defined above in formula I; and each occurrence of R^{1c′} is independently —H. —(C1—C6)-aliphatic (such as —(C1—C6)-alkyl) or (C3-C6)-cycloalkyl; preferably, each occurrence of R^{1c′} is independently —H or —(C1—C6)-aliphatic (such as —(C1—C6)-alkyl).

In some embodiments of prodrug-IC1 or prodrug-IC2, at least one occurrence of R^{1c} is an alkyl group, such as methyl, ethyl, isopropyl or t-butyl). In some embodiments of prodrug-IC1 or prodrug-IC2. at least one occurrence of R^{1c} is —H. In certain embodiments of prodrug-IC1 or prodrug-IC2, at least one occurrence of R^{1c′} is a -(C1-C6)-alkyl group, such as methyl, ethyl or isopropyl. In some embodiments of prodrug-IC1 or prodrug-IC2, all the occurrences of R^{1c} are the same In some embodiments, all the occurrences of R^{1c′} are the same

In certain embodiments, the prodrug of the present disclosure for use in treating eye disorders has the formula:

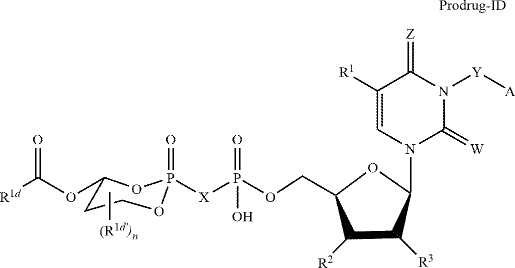

or a salt thereof, wherein: A, X, Y, Z, W, R¹, R² and R³ are as defined above in formula I; R^{1d} is a group selected from aliphatic (such as —(C1—C6)-alkyl), heterocyclyl, cycloalkyl, cycloalkenyl, aryl and heteroaryl, wherein said aliphatic, heterocyclyl, cycloalkyl, cycloalkenyl, aryl or heteroaryl is unsubstituted or substituted with at least one R⁷ as defined above in formula I; n is 0-5, preferably 0-2, most preferably 0; and each occurrence of R^{1d′} is independently selected from —H and R⁷ as defined above in formula I.

In some embodiments of prodrug-ID, R^{1d} is an alkyl group, such as methyl, ethyl, isopropyl or t-butyl. In other embodiments of prodrug-ID, R^{1d} is an optionally substituted phenyl. In certain embodiments, n is 0. In preferred embodiments where n is 1 or 2, all R^{1d′} are attached to the carbon of the ring distal to the carbon bearing R^{1d}CO₂.

In certain embodiments, the prodrug of the present disclosure for use in treating eye disorders has the formula:

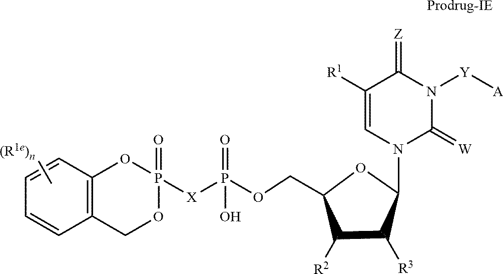

or a salt thereof, wherein: A, X, Y, Z, W, R¹, R² and R³ are as defined above in formula I; n is 0-4; and each occurrence of R^{1e} is independently selected from —H and R⁷ as defined above in formula I.

In some embodiments of prodrug-IE, at least one occurrence of R^{1e} is a —(C1—C6)-alkyl group, such as methyl, ethyl, isopropyl or t-butyl. In some embodiments of prodrug-IE, at least one occurrence of R^{1e} is halogen, preferably —F or —C1. In certain embodiments, n is 1. In certain embodiments of prodrug-IE, n is 1 and R¹ is methyl.

In certain embodiments, the prodrug of the present disclosure for use in treating eye disorders has the formula:

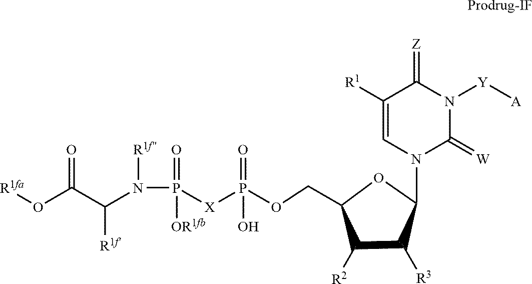

or a salt thereof, wherein: A, X, Y, Z, W, R² and R³ are as defined above in formula I; R^{1fa} and R^{1fb} each independently is a group selected from —H, aliphatic (such as —(C1—C6)-alkyl), heterocyclyl, cycloalkyl, cycloalkenyl, aryl and heteroaryl, wherein said aliphatic, heterocyclyl, cycloalkyl, cycloalkenyl, aryl or heteroaryl is unsubstituted or substituted with at least one R⁷ as defined above in formula 1; and R^{1f′} and R^{1f″} each independently is a group selected from —H, —(C1—C6)-aliphatic (such as —(C1—C6)-alkyl) and —(C3—C6)-cycloalkyl; preferably. R^{1f′} and R^{1f″} each independently is a group selected from —H or — (C1—C6)-aliphatic (such as —(C1—C6)-alkyl).

In some embodiments of prodrug-IF, R^{1fa} is an alkyl group, such as methyl, ethyl, isopropyl or t-butyl. In some embodiments of prodrug-IF, R^{1fb} is an optionally substituted phenyl. In some embodiments of prodrug-IF. R^{1f′} is —H. In certain embodiments of prodrug-IF. R^{1f′} is a —(C1—C6)-alkyl group, such as methyl, ethyl or isopropyl. In some embodiments of prodrug-IF. R^{1F′} is —H. In certain embodiments of prodrug-IF, R^{1f′} is a —(C1—C6)-alkyl group, such as methyl, ethyl or isopropyl, and R^{1f′} is —H.

In certain embodiments, the prodrug of the present disclosure for use in treating eye disorders has the formula:

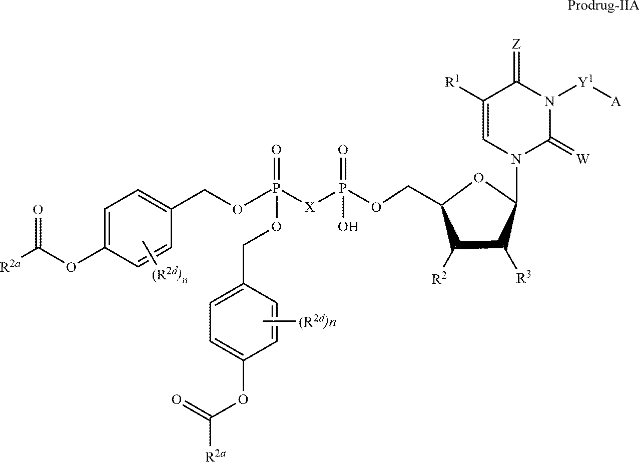

or a salt thereof, wherein: X, Y¹, Z, W, R² and R³ are as defined above in formula II; A is selected from a phenyl group that is unsubstituted or substituted with at least one (C1-C5)-aliphatic group or halogen: a naphthalene group: a 5- to 10-membered heteroaryl group having up to 5 heteroatoms independently selected from N, O and S, and a 3- to 10-membered non-aromatic ring having up to 5 heteroatoms independently selected from N, O, S, SO, or SO₂; wherein A is optionally further substituted with one or more R⁴; each n is independently 0-4; each occurrence of R^{2a} is a group independently selected from aliphatic (such as —(C1—C6)-alkyl), heterocyclyl, cycloalkyl, cycloalkenyl, aryl and heteroaryl, wherein said aliphatic, heterocyclyl, cycloalkyl, cycloalkenyl, aryl or heteroaryl is unsubstituted or substituted with at least one R⁴ as defined above in formula II; and each occurrence of R^{2a′} is independently selected from —H and R⁴ as defined above in formula II.

In some embodiments of prodrug-IIA, at least one R^{2a} is an alkyl group, such as methyl, ethyl, isopropyl or t-butyl In some embodiments of prodrug-IIA, at least one R^{2a} is an optionally substituted phenyl In preferred embodiments, n is 0. In certain embodiments of prodrug-IIA, both occurrences of R^{2a} are the same.

In certain embodiments, the prodrug of the present disclosure for use in treating eye disorders has the formula:

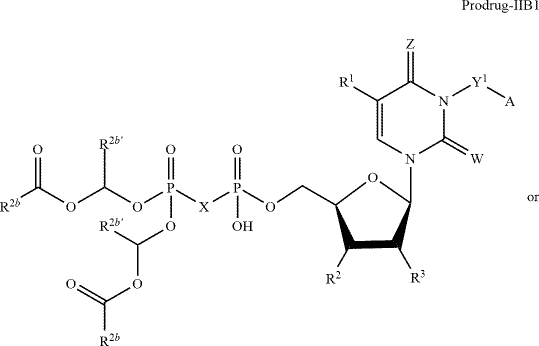

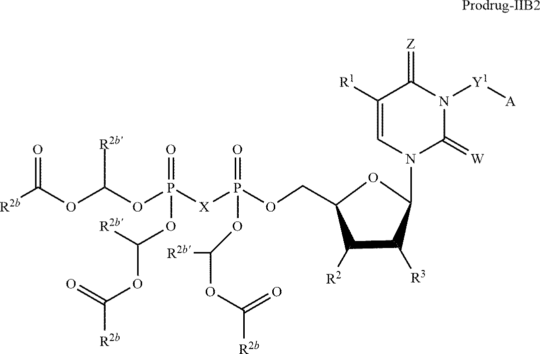

or a salt thereof, wherein: X, Y¹, Z, W, R² and R³ are as defined above in formula II; A is selected from: a phenyl group that is unsubstituted or substituted with at least one (C1-C5)-aliphatic group or halogen; a naphthalene group: a 5- to 10-membered heteroaryl group having up to 5 heteroatoms independently selected from N, O and S: and a 3- to 10-membered non-aromatic ring having up to 5 heteroatoms independently selected from N. O, S, SO. or SO₂; wherein A is optionally further substituted with one or more R⁴; each occurrence of R^{2b} is a group independently selected from aliphatic (such as —(C1—C6)-alkyl), heterocyclyl, cycloalkyl, cycloalkenyl, aryl and heteroaryl, wherein said aliphatic, heterocyclyl, cycloalkyl, cycloalkenyl, aryl or heteroaryl is unsubstituted or substituted with at least one R⁴ as defined above in formula II; and each occurrence of R^{2b′} is independently —H, —(C1—C6)-aliphatic (such as —(C1—C6)-alkyl) or (C3-C6)-cycloalkyl; preferably, each occurrence of R^{2b′} is independently —H or —(C1—C6)-aliphatic (such as —(C1—C6)-alkyl).

In some embodiments of prodrug-IIB 1 or prodrug-IIB2, at least one occurrence of R^{2b} is an alkyl group, such as methyl, ethyl, isopropyl or t-butyl. In some embodiments of prodrug-IIB1 or prodrug-IIB2, at least one occurrence of R^{2b′} is —H. In certain embodiments of prodrug-IIBI or prodrug-IIB2, at least one occurrence of R^{2b′} is a —(C1—C6)-alkyl group, such as methyl, ethyl or isopropyl. In some embodiments of prodrug-IIB1 or prodrug-IIB2, all the occurrences of R^{2b} are the same. In some embodiments, all the occurrences of R^{2b′} are the same.

In certain embodiments, the prodrug of the present disclosure for use in treating eye disorders has the formula:

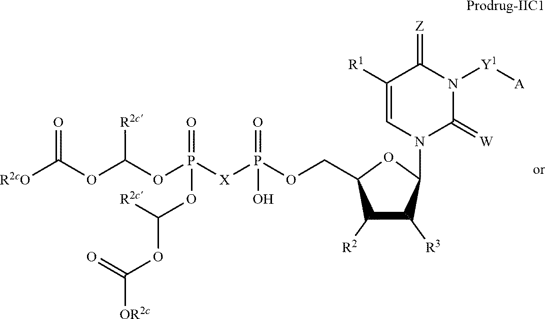

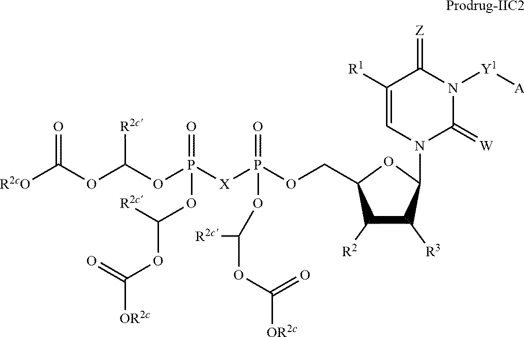

or a salt thereof, wherein: X, Y¹, Z. W, R² and R³ are as defined above in formula II; A is selected from a phenyl group that is unsubstituted or substituted with at least one (C1 -C5)-aliphatic group or halogen; a naphthalene group; a 5- to 10-membered heteroaryl group having up to 5 heteroatoms independently selected from N, O and S; and a 3- to 10-membered non-aromatic ring having up to 5 heteroatoms independently selected from N, O, S, SO, or SO₂; wherein A is optionally further substituted with one or more R⁴; each occurrence of R^{2c} is a group independently selected from aliphatic (such as —(C1—C6)-alkyl), heterocyclyl, cycloalkyl, cycloalkenyl, aryl and heteroaryl, wherein said aliphatic, heterocyclyl, cycloalkyl, cycloalkenyl, aryl or heteroaryl is unsubstituted or substituted with at least one R⁴ as defined above in formula II; and each occurrence of R^{2c′} is independently — H, —(C1—C6)-aliphatic (such as —(C1—C6)-alkyl) or (C3-C6)-cycloalkyl; preferably, each occurrence of R^{2c′} is independently —H or —(C1—C6)-aliphatic (such as —(C1—C6)-alkyl).

In some embodiments of prodrug-IIC1 or prodrug-IIC2, at least one occurrence of R^{2c} is an alkyl group, such as methyl, ethyl, isopropyl or t-butyl. In some embodiments of prodrug-IIC1 or prodrug-IIC2, at least one occurrence of R^{2c′} is —H. In certain embodiments of prodrug-IIC1 or prodrug-IIC2, at least one occurrence of R^{2c′} is a —(C1—C6)-alkyl group, such as methyl, ethyl or isopropyl. In some embodiments of prodrug-IIC1 or prodrug-IIC2, all the occurrences of R^{2c} are the same. In some embodiments, all the occurrences of R^{2c′} are the same.

In certain embodiments, the prodrug of the present disclosure for use in treating eye disorders has the formula:

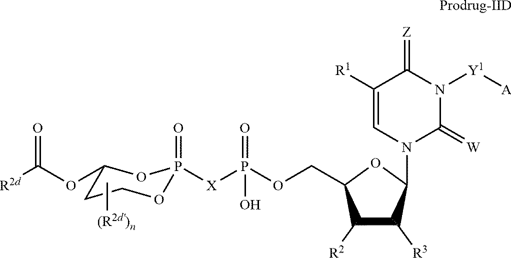

or a salt thereof, wherein X. Y¹, Z, W, R¹⁻, R² and R³ are as defined above in formula II; A is selected from: a phenyl group that is unsubstituted or substituted with at least one (C1-(5)-aliphatic group or halogen; a naphthalene group; a 5- to 10-membered heteroaryl group having up to 5 heteroatoms independently selected from N, O and S; and a 3- to 10-membered non-aromatic ring having up to 5 heteroatoms independently selected from N, O, S. SO, or SO₂; wherein A is optionally further substituted with one or more R⁴; R^{2d} is a group selected from aliphatic (such as —(C1—C6)-alkyl), heterocyclyl, cycloalkyl, cycloalkenyl, aryl and heteroaryl, wherein said aliphatic, heterocyclyl, cycloalkyl, cycloalkenyl, aryl or heteroaryl is unsubstituted or substituted with at least one R⁴ as defined above in formula II; n is 0-5, preferably 0-2. most preferably 0; and each occurrence of R^{2d′} is independently selected from —H and R⁴ as defined above in formula II.

In some embodiments of prodrug-IID, R^{2d} is an alkyl group, such as methyl, ethyl, isopropyl or t-butyl. In other embodiments of prodrug-IID, R^{2d} is an optionally substituted phenyl. In certain embodiments, n is 0. In preferred embodiments where n is 1 or 2, all R^{2d′} are attached to the carbon of the ring distal to the carbon bearing R^{2d}CO₂

In certain embodiments, the prodrug of the present disclosure for use in treating eye disorders has the formula:

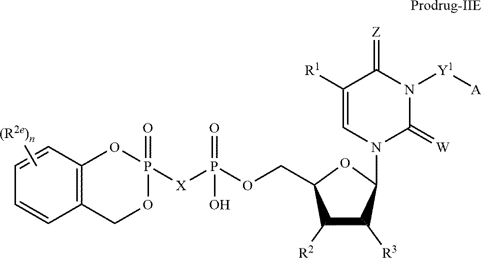

or a salt thereof, wherein: X. Y¹, Z, W, R¹, R² and R³ are as defined above in formula II; A is selected from: a phenyl group that is unsubstituted or substituted with at least one (C1-C5)-aliphatic group or halogen; a naphthalene group; a 5- to 10-membered heteroaryl group having up to 5 heteroatoms independently selected from N, O and S; and a 3- to 10-membered non-aromatic ring having up to 5 heteroatoms independently selected from N, O, S. SO, or SO₂; wherein A is optionally further substituted with one or more R⁴; n is 0-4: and each occurrence of R^{2e} is independently selected from —H and R⁴as defined above in formula II.

In some embodiments of prodrug-IIE, at least one occurrence of R^{2e} is a —(C1-C6)-alkyl group; such as methyl, ethyl, isopropyl or t-butyl. In some embodiments of prodrug-IIE, at least one occurrence of R^{2e} is halogen, preferably, —F or —Cl. In certain embodiments, n is 1. In some embodiments of prodrug-IIE, n is 1 and R^{2e} is methyl.

In certain embodiments, the prodrug of the present disclosure for use in treating eye disorders has the formula:

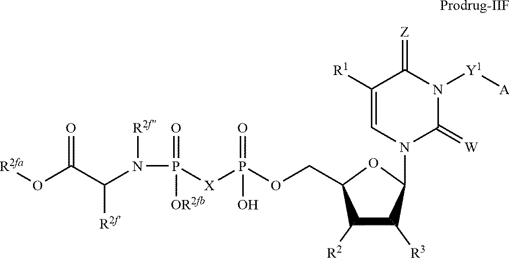

or salt thereof, wherein: X. Y¹, Z, W, R² and R³ are as defined above in formula II; A is selected from a phenyl group that is unsubstituted or substituted with at least one (C I-C5)-aliphatic group or halogen: a naphthalene group: a 5- to 10-membered heteroaryl group having up to 5 heteroatoms independently selected from N. O and S; and a 3- to 10-membered non-aromatic ring having up to 5 heteroatoms independently selected from N, O, S. SO, or SO₂; wherein A is optionally further substituted with one or more R⁴; R^{2fa} and R^{2fb} each independently is a group selected from —H, aliphatic (such as —(C1—C6)-alkyl), heterocyclyl, cycloalkyl, cycloalkenyl, aryl and heteroaryl, wherein said aliphatic, heterocyclyl, cycloalkyl, cycloalkenyl, aryl or heteroaryl is unsubstituted or substituted with at least one R⁴ as defined above in formula II; and R^{2f′} and R^{2f″} each independently is a group selected from —H, —(C1—C6)-aliphatic (such as —(C1—C6)-alkyl) and —(C3—C6)-cycloalkyl; preferably, R^{2f′} and R^{2f″} each independently is a group selected from —H or — (C1—C6)-aliphatic (such as —(C1—C6)-alkyl).

In some embodiments of prodrug-IIF, R^{2fa} is an alkyl group, such as methyl, ethyl, isopropyl or t-butyl. In some embodiments of prodrug-IIF. R^{2fb} is an optionally substituted phenyl. In some embodiments of prodrug-IIF, R^{2f′} is —H. In certain embodiments of prodrug-IIF, R^{2f′} is a —(C1—C6)-alkyl group, such as methyl, ethyl or isopropyl. In some embodiments of prodrug-IIF, R^{2f″} is —H. In certain embodiments of prodrug-IIF, R^{2f′} is a (C1-C6)-alkyl group, such as methyl, ethyl or isopropyl, and R^{2r} is —H.

For a compound of the formula I or II for use in treating eye disorders:

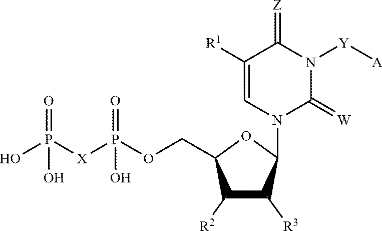

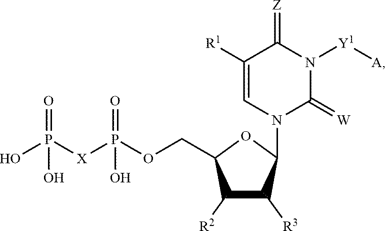

representative prodrugs include:

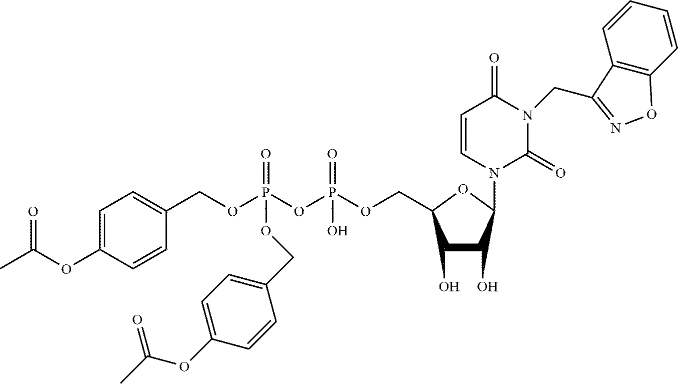

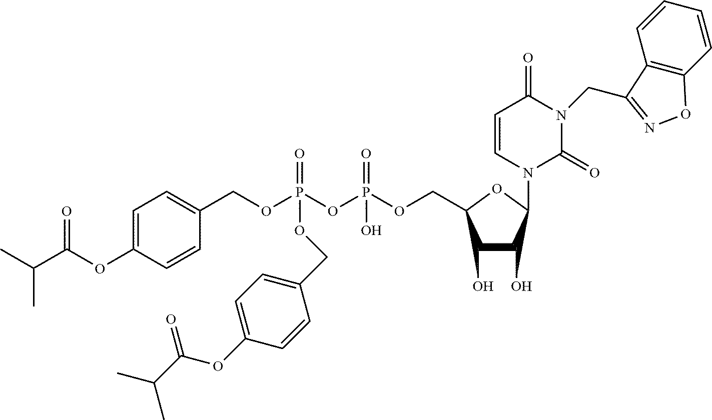

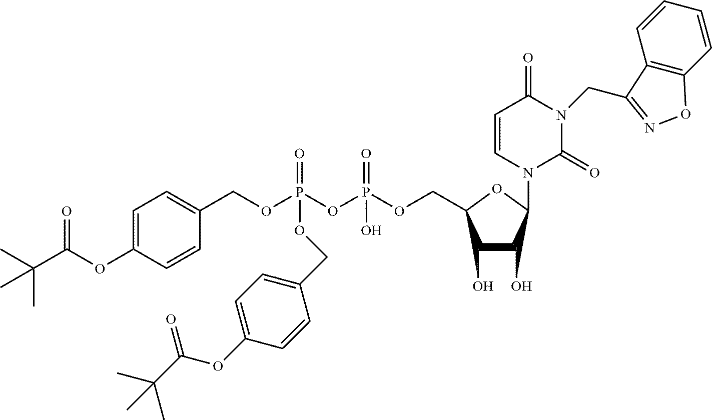

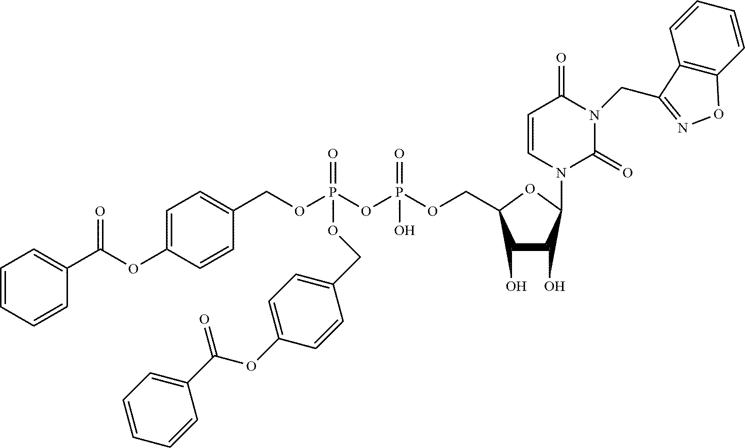

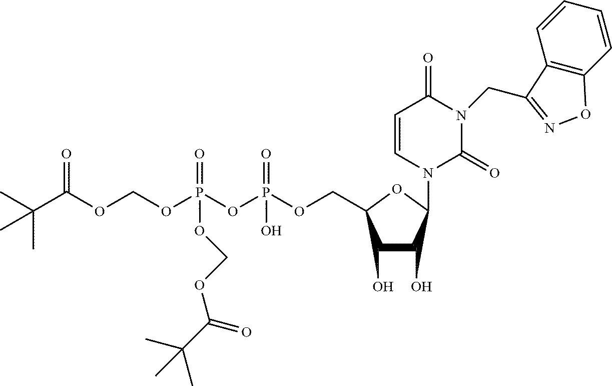

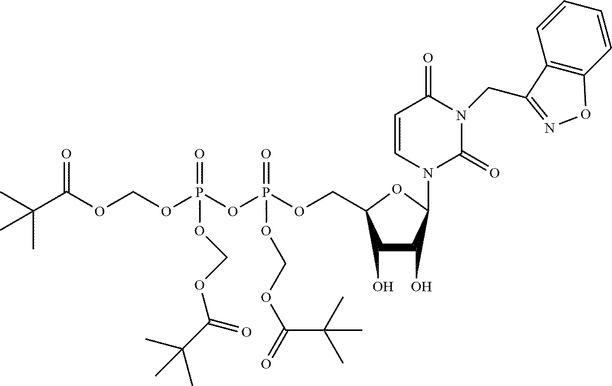

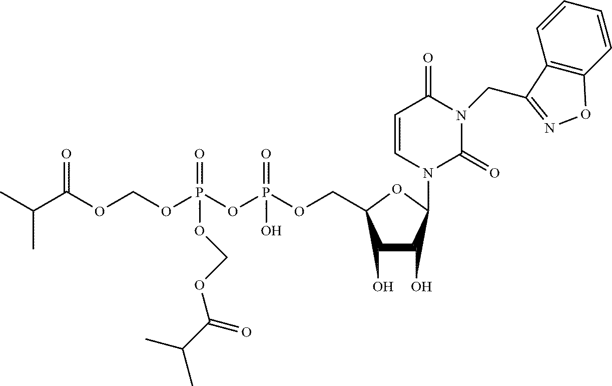

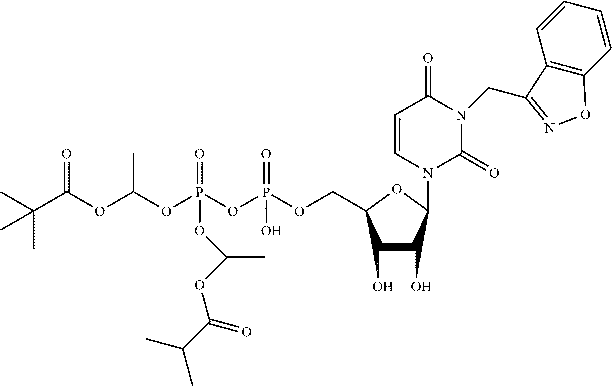

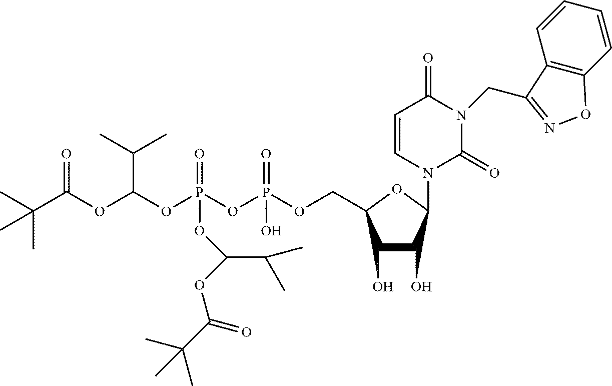

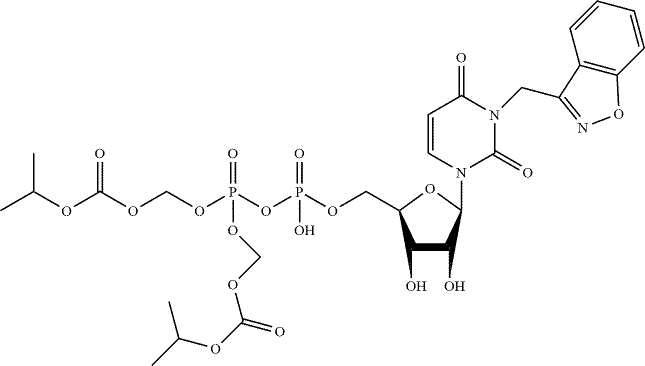

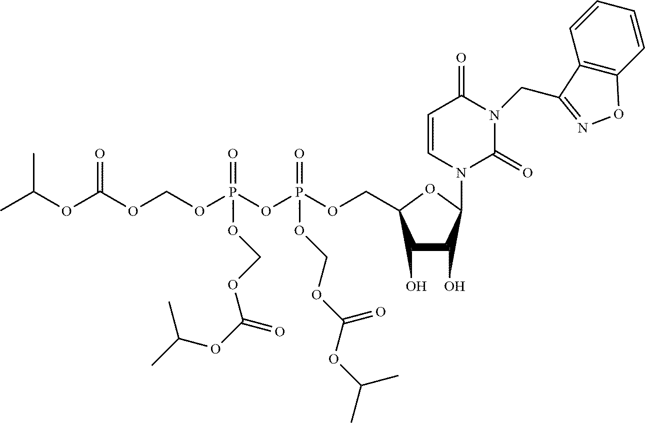

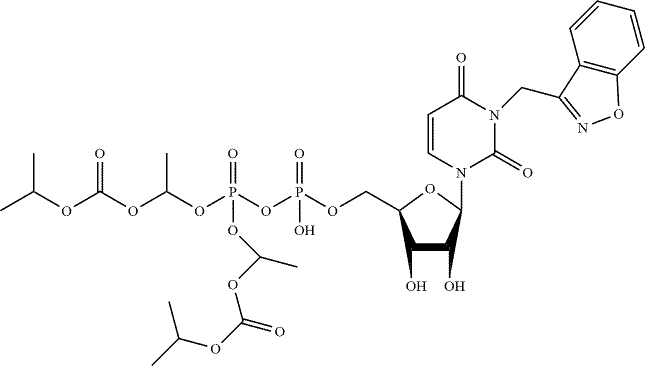

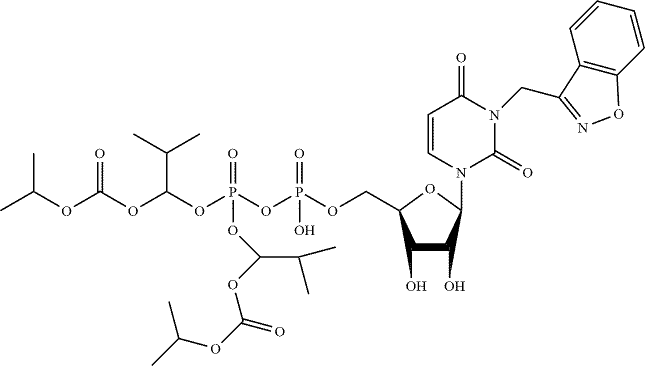

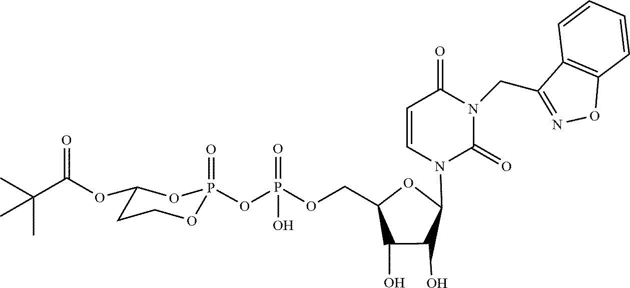

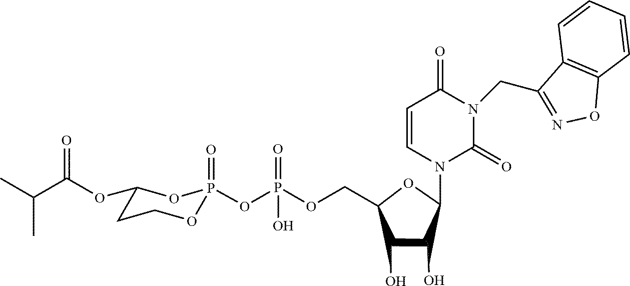

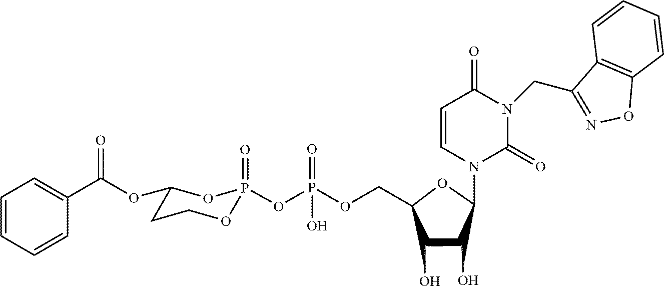

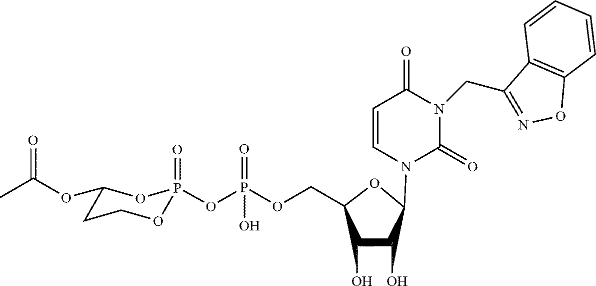

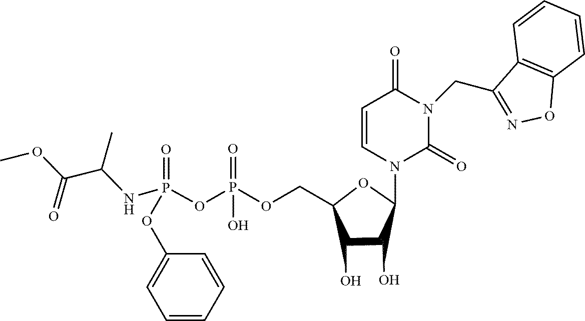

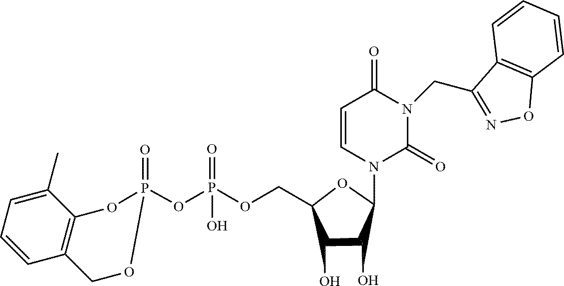

or salts thereof. In some embodiments of the prodrug of the present disclosure, the salt is a sodium salt.

In another embodiment, the present disclosure provides a pharmaceutical composition comprising a pharmaceutically acceptable carrier and a prodrug of a compound of formula I or II or pharmaceutically acceptable salt form thereof for use in treating a disorder of the eye.

The disclosure contemplates that any one or more of the foregoing aspects and embodiments can be combined with each other and/or with any of the embodiments or features provided below.

In certain aspects, the compounds, or salts and/or prodrugs thereof, and compositions as described herein can be used to treat patients suffering from P₂Y₆ receptor-related conditions, such as retinal degenerative diseases, and traumatic or mechanical injury to the retina, and optic nerve. Many of these, as well as other conditions described herein, are characterized by a level of vision loss and decreased visual acuity. Visual acuity generally refers commonly refers to the clarity of vision. Visual acuity is dependent on optical and neural factors, such as the sharpness of the retinal focus in the eye, the health and functioning of the retina, the health and functioning of the optic nerve and the interpretative faculty of the brain.

Retinal degenerative diseases typically involves the loss of neurons in the retina which may be due to the atrophy or death. Neurodegenerative diseases can evolve gradually, after a long period of apparent normal retinal function, due to progressive degeneration (i.e., nerve cell dysfunction and death). Alternatively, neurodegenerative diseases can have a quick onset, such as those associated with ischemic and radiation injury and exposure to retinal toxins. The actual onset of brain degeneration may precede clinical expression by many years. Examples of neurodegenerative diseases include, but are not limited to glaucoma, age-related macular degeneration, anterior ischemic optic neuropathy, diabetic retinopathy and vascular occlusions. P₂Y₆ receptor modulating compounds, or salts and/ or prodrugs can be used to treat these disorders and others as described below.

P₂Y₆ receptor modulating compounds or prodrugs thereof, will be used to treat glaucoma by limiting the death of retinal ganglion cells in glaucomatous individuals. Glaucoma is a family of eye diseases, often characterized by elevated intraocular pressure, that results in progressive loss of retinal ganglion cells and vision loss. The most common type is open-angle glaucoma. Cell death in glaucoma can involves the direct injury to the retinal ganglion cell axons at the lamina cribrosa, ischemic injury and or glial activation. The expression of P₂Y receptors in the trabecular meshwork and the observation that P₂Y agonists lower IOP, indicate that these compounds will also lower IOP. P₂Y₆ agonists will mitigate one or more of the initiating events associated with retinal ganglion cell loss in glaucoma.

P₂Y₆ receptor modulating compounds or prodrugs thereof, will be used to treat age-related macular degeneration (AMD) by slowing the death and or dysfunction of retinal photoreceptors and retinal pigment epithelium (RPE). There are two types of AMD: wet and dry. The less common wet from results from growth of blood vessels under the retina. The dry form is the most common type of AMD and results from the slow deterioration of the of RPE and photoreceptor cells. P₂Y₆ receptors and been identified in the RPE and photoreceptors. The activation of these receptors will improve the function of these cells and limit vision loss in individuals diagnosed with AMD.

P₂Y₆ receptor modulating compounds or prodrugs thereof, will be used to treat trauma to the retina, including, physical injury (including surgical intervention), or environmental trauma (e.g., visible light damage, x-ray, etc.). In certain embodiments, compounds, or salts and/or prodrugs thereof, of the present disclosure may be used to treat traumatic retinal injury, such as to improve visual acuity.

P₂Y₆ receptor modulating compounds or prodrugs thereof, will be used to treat ocular uveitis. Uveitis is a general term describing a family of inflammatory diseases that produces swelling and destroys ocular tissues. The term “uveitis” is used as these inflammatory disorders mainly affect the vascular parts of the eye called the uvea. However, uveitis can impact nonvascular portions of the eye such as the lens and cornea. These diseases can also the retina, optic nerve, and vitreous, reducing visual acuity. Uveitis may be caused by events occurring locally in the eye or part of systemic inflammatory diseases. In Uveitis there is often high morbidity and mortality with no effective immunomodulatory treatment to prevent the overwhelming synthesis of proinflammatory mediators. Currently long-term corticosteroid treatment is the therapeutic mainstay, with serious potential complications. Hence, there is an unmet need for alternative anti-inflammatory treatments. P₂Y₆ receptor agonists have been found to suppress the actions of inflammatory cytokines as well as regulating immune cell function. The administration of P₂Y₆ agonists and subsequent activation of P₂Y receptors will suppress inflammatory responses in the eye and limit vision loss in individuals diagnosed with uveitis.

P₂Y₆ receptor modulating compounds or prodrugs thereof, will be used to treat dry eye conditions. Dry eye is associated with dysfunction of the mucous membranes and lacrimal gland of the eye. P₂Y receptors have been identified in both tissues. The administration of P₂Y₆ agonists and subsequent activation of P₂Y receptors will enhance tear film production and mucous secretions in the eye improving comfort and visual acuity of affected individuals.

The P₂Y receptor modulating compounds may be administered to a patient needing treatment for an disorder in combination with a opthalmically-acceptable vehicle or carrier. Other components, which may be included in the carrier components include, without limitation, buffer components, tonicity components, preservative-components, pH adjustors, components commonly found in artificial tears, such as one or more electrolytes, and the like and mixtures thereof. In one very useful embodiment the carrier component includes at least one of the following: an effective amount of a buffer component; an effective amount of a tonicity component; an effective amount of is a preservative component; and water.

These additional components preferably are ophthalmically acceptable and can be chosen from materials which are conventionally employed in ophthalmic compositions, for example, compositions used to treat eyes afflicted with dry eye syndrome or another eye disorder, artificial tear formulations and the like.

Acceptable effective concentrations for these additional components in the compositions of the invention are readily apparent to the skilled practitioner.

Said compounds may be administered, alone, or in combination with pharmaceutically acceptable substances including buffer solutions, for example phosphate buffered saline, or inert carrier compounds, glycerols, mineral oils or similar substances to the ocular surface of the eye.

The dosage of the above lipid compounds is optimized according to the formulation and method of delivery and the mode of administration is determined by conventional protocols and effectively treats eye disorder symptoms in humans.

The P₂Y receptor modulating compounds may be utilized as a vehicle for topical administration of a therapeutic medicament. In particular, the P₂Y receptor modulating compound-containing vehicle is used to deliver any desired therapeutic agent, or combination of therapeutic agents, including an antibiotic agent, an antiviral agent, an antifungal agent, an anti-cancer agent, an antiglaucoma agent, an antiinflammatory agent, secretagogues exemplified by but not limited to agents that promote lacrimation, salivation or stimulation of release of soluble mucins and or expression of cell associated mucins that promote wettability and/or lubricity of mucosal surfaces, an analgesic, an immunomodulatory agent, a macro-molecule, or a mixture thereof.

Therapeutic agents that are used in the method of the present invention include, but are not limited to NMDA antagonists, antihistamines, antiparasitics, miotics, sympathomimetics, anticholinergics, local anesthetics, amoebicidals, trichomonocidals, mydriatics, carbonic anhydrase inhibitors, ophthalmic diagnostic agents, ophthalmic agents used as adjuvants in surgery, chelating agents, antineoplastics, diagnostics, adrenergic anesthetics, beta blockers, alpha-2-agonists, cycloplegics, prostaglandins, ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, antihypertensives, pressors, antibacterials, antivirals, antifungals, antiprotozoals, anti-infectives, antitumor agents, antimetabolites, antiangiogenic agents, tyrosine kinase inhibitors, antibiotics such as aminoglycosides such as gentamycin, kanamycin, neomycin, and vancomycin; amphenicols such as chloramphenicol; cephalosporins, such as cefazolin HCl; penicillins such as ampicillin, penicillin, carbenicillin, oxycillin, methicillin; lincosamides such as lincomycin; polypeptide antibiotics such as polymixin and bacitracin; tetracyclines such as tetracycline; quinolones such as ciproflaxin, etc.; sulfonamides such as chloramine T; and sulfones such as sulfanilic acid as the hydrophilic entity, anti-viral drugs, e.g. acyclovir, gancyclovir, vidarabine, azidothymidine, dideoxyinosine, dideoxycytosine, dexamethasone, ciproflaxin, water soluble antibiotics, such as acyclovir, gancyclovir, vidarabine, azidothymidine, dideoxyinosine, dideoxycytosine; epinephrine; isoflurphate; adriamycin; bleomycin; mitomycin; ara-C; actinomycin D; scopolamine; and the like, analgesics, such as codeine, morphine, keterolac, naproxen, etc., an anesthetic, e.g. lidocaine; .beta.-adrenergic blocker or .beta.-adrenergic agonist, e.g. ephidrine, epinephrine, etc.; aldose reductase inhibitor, e.g. epalrestat, ponalrestat, sorbinil, tolrestat; antiallergic, e.g. cromolyn, beclomethasone, dexamethasone, and flunisolide; colchicine; antiamebic agents, e.g. chloroquine and chlortetracycline; and antifungal agents, e.g. amphotericin, etc., anti-angiogenesis compounds such as anecortave acetate, anti-glaucoma agents, such as brimonidine, acetozolamide, bimatoprost, Timolol, mebefunolol; memantine; alpha-2 adrenergic receptor agonists; 2ME2; anti-neoplastics, such as vinblastine, vincristine, interferons; alpha., beta. and .gamma., antimetabolites, such as folic acid analogs, purine analogs, and pyrimidine analogs; immunosuppressants such as azathiprine, cyclosporine and mizoribine; miotic agents, such as carbachol, mydriatic agents such as atropine, etc., protease inhibitors such as aprotinin, camostat, gabexate, vasodilators such as bradykinin, etc., and various growth factors, such epidermal growth factor, basic fibroblast growth factor, nerve growth factors, and the like, including derivatives thereof and mixtures thereof.

The effective amount of said P₂Y receptor modulating compounds are preferably administered as a vehicle is specified by routine methods and may be combined with pharmaceutically acceptable substances utilized in ophthalmic vehicles, including buffer solutions, for example phosphate buffered saline, or inert carrier compounds, glycerols, mineral oils or similar substances. The dosage of said P₂Y modulating compound is optimized according to the formulation and method of delivery and the mode of administration are determined by conventional protocols to effectively treat the relevant eye disorder symptoms in humans.

In some preferred embodiments, the P₂Y receptor modulating compound-containing vehicle is administered topically, e.g. as an eye drop, to provide “artificial tears.”

In some preferred embodiments, the P₂Y receptor modulating compound-containing vehicle is used in a method of treating a patient suffering from “dry eye” and related ocular disorders to provide improved stability of the tear film of a patient in need of said treatment.

In some preferred embodiments, the P₂Y receptor modulating compound may be utilized as a vehicle for topical administration of a therapeutic medicament. In some preferred embodiments, the P₂Y modulating compound-containing vehicle is used to deliver any desired therapeutic agent, or combination of therapeutic agents, including an antibiotic agent, an antiviral agent, an antifungal agent, an anti-cancer agent, an antiglaucoma agent, an antiinflammatory agent, secretagogues exemplified by but not limited to agents that promote lacrimation, salivation or stimulation of release of soluble mucins and or expression of cell associated mucins that promote wettability and/or lubricity of mucosal surfaces, an analgesic, an immunomodulatory agent, a macro-molecule, or a mixture thereof.

Therapeutic agents that are used in the method of the present invention include, but are not limited to NMDA antagonists, antihistamines, antiparasitics, miotics, sympathomimetics, anticholinergics, local anesthetics, amoebicidals, trichomonocidals, mydriatics, carbonic anhydrase inhibitors, ophthalmic diagnostic agents, ophthalmic agents used as adjuvants in surgery, chelating agents, antineoplastics, diagnostics, adrenergic anesthetics, beta blockers, alpha-2-agonists, cycloplegics, prostaglandins, ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, antihypertensives, pressors, antibacterials, antivirals, antifungals, antiprotozoals, anti-infectives, antitumor agents, antimetabolites, antiangiogenic agents, tyrosine kinase inhibitors, antibiotics such as aminoglycosides such as gentamycin, kanamycin, neomycin, and vancomycin; amphenicols such as chloramphenicol; cephalosporins, such as cefazolin HCl; penicillins such as ampicillin, penicillin, carbenicillin, oxycillin, methicillin; lincosamides such as lincomycin; polypeptide antibiotics such as polymixin and bacitracin; tetracyclines such as tetracycline; quinolones such as ciproflaxin, etc.; sulfonamides such as chloramine T; and sulfones such as sulfanilic acid as the hydrophilic entity, anti-viral drugs, e.g. acyclovir, gancyclovir, vidarabine, azidothymidine, dideoxyinosine, dideoxycytosine, dexamethasone, ciproflaxin, water soluble antibiotics, such as acyclovir, gancyclovir, vidarabine, azidothymidine, dideoxyinosine, dideoxycytosine; epinephrine; isoflurphate; adriamycin; bleomycin; mitomycin; ara-C; actinomycin D; scopolamine; and the like, analgesics, such as codeine, morphine, keterolac, naproxen, etc., an anesthetic, e.g. lidocaine; .beta.-adrenergic blocker or .beta.-adrenergic agonist, e.g. ephidrine, epinephrine, etc.; aldose reductase inhibitor, e.g. epalrestat, ponalrestat, sorbinil, tolrestat; antiallergic, e.g. cromolyn, beclomethasone, dexamethasone, and flunisolide; colchicine; antiamebic agents, e.g. chloroquine and chlortetracycline; and antifungal agents, e.g. amphotericin, etc., anti-angiogenesis compounds such as anecortave acetate, anti-glaucoma agents, such as brimonidine, acetozolamide, bimatoprost, Timolol, mebefunolol; memantine; alpha-2 adrenergic receptor agonists; 2ME2; anti-neoplastics, such as vinblastine, vincristine, interferons; alpha., beta. and .gamma., antimetabolites, such as folic acid analogs, purine analogs, and pyrimidine analogs; immunosuppressants such as azathiprine, cyclosporine and mizoribine; miotic agents, such as carbachol, mydriatic agents such as atropine, etc., protease inhibitors such as aprotinin, camostat, gabexate, vasodilators such as bradykinin, etc., and various growth factors, such epidermal growth factor, basic fibroblast growth factor, nerve growth factors, and the like, including derivatives thereof and mixtures thereof.

The present invention is not to be limited in scope by the exemplified embodiments, which are only intended as illustrations of specific aspects of the invention. Although there is described hereinabove a specific method of treating eye disorders with P₂Y receptor modulating compounds in accordance with the present invention for the purpose of illustrating the manner in which the invention can be used to advantage, it will be appreciated that the invention is not limited thereto. For example, the methods and compositions of the present invention may be used to treat other ocular conditions and disorders. Accordingly, any and all variations and modifications which may occur to those skilled in the art are to be considered to be within the scope and spirit of the invention as defined in the appended claims.

What is claimed is:

1. A method of treating an eye disorder in a subject in need thereof comprising administering an effective amount of a P₂Y receptor modulating compound.

2. The method of claim 1, wherein the eye disorder is selected from the group consisting of retinal neurodegenerations, including glaucoma, age-related macular degeneration and traumatic retinal injury, posterior and anterior uveitis of the eye, and dry eye disorders.

3. The method of any one of claims 1 to 2, wherein the P₂Y receptor modulating compound is a P₂Y₆ receptor modulating compound.

4. The method of any one of claims 1 to 3, wherein the P₂Y receptor modulating compound is a uridine diphosphate derivative or prodrug thereof.

5. The method of claim 4, wherein the uridine diphosphate derivative is a compound of formula I or a salt or a prodrug thereof: 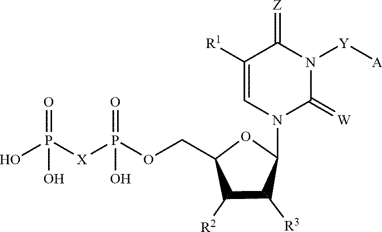
I
or a salt thereof, wherein:
A is 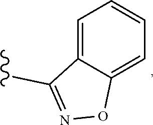
wherein A is optionally substituted with one or more R⁷;
X is independently selected from —O—, —S—, —N(R⁵)— and a (C1-C3)-aliphatic group independently and optionally substituted with one or more R⁴;
Y is a bond or a (C1-C5)-aliphatic group independently and optionally substituted with one or more R⁴;
Z and W are each independently selected from ═O, ═S. ═N(R⁵), and ═NOR⁵;
R¹ is selected from: —H—, halogen. —OR⁵, —CN, —CF₃, —OCF₃ and a (C1-C6)-aliphatic group optionally substituted with one or more R⁷;
R² and R³ are each independently selected from —OR⁵, —SR⁵, —NR⁵R⁶, —OC(O)R⁵, —OC(O)NR⁵R⁶, and —OC(O)OR⁵;
each occurrence of R⁴ is independently selected from halogen, —OR⁵, —NO₂, —CN, —CF₃, —OCF₃, —R⁵, 1,2-methylenedioxy, 1,2-ethylenedioxy. —N(R⁵)₂, —SR⁵, —SOR⁵, —SO₂R⁵, —SO₂N(R⁵)₂, —SO₃R⁵, —C(O)R⁵, —C(O)C(O)R⁵, —C(O)CH₂C(O)R⁵, —C(S)R⁵, —C(S)OR⁵, —C(O)OR⁵, —C(O)C(O)OR⁵, —C(O)C(O)N(R⁵)₂, —OC(O)R⁵, —C(O)N(R⁵)₂, —OC(O)N(R⁵)₂, —C(S)N(R⁵)₂, —(CH₂)_{0—2}NHC(O)R⁵, —N(R⁵)N(R⁵)COR⁵, —N(R⁵)N(R⁵)C(O)OR⁵, —N(R⁵)N(R⁵)CON(R⁵)₂, —N(R⁵)SO₂R⁵, —N(R⁵)SO₂N(R⁵)₂, —N(R⁵)C(O)OR⁵, —N(R⁵)C(O)R⁵, —N(R⁵)C(S)R⁵, —N(R⁵)C(O)N(R⁵)₂, —N(R⁵)C(S)N(R⁵)₂, —N(COR⁵)COR⁵, —N(OR⁵)R⁵, —C(═NH)N(R⁵)₂, —C(O)N(OR⁵)R⁵, —C(═NOR⁵)R⁵, —OP(O)(OR⁵)₂, —P(O)(R⁵)₂, —P(O)(OR⁵)₂, or —P(O)(H)(OR⁵);
each occurrence of R⁵ is independently selected from: H—, {C1-C12)-aliphatic-. (C3-C10)-cycloalkyl-or-cycloalkenyl-, [(C3-C10)-cycloalkyl or -cycloalkenyl-]-(C1-C12)-aliphatic-, (C6-C10)-aryl-, (C6-C10)-aryl-(C1-C12)aliphatic-, (C3-C10)-heterocyclyl-, (C6-C10)-heterocyclyl-(C1-C12)aliphatic-, (C5-C10)-heteroaryl-, and (C5-C10)-heteroaryl-(C1-C12)-aliphatic-; wherein two R⁵ groups bound to the same atom optionally form a 3- to 10-membered aromatic or non-aromatic ring having up to 3 heteroatoms independently selected from N, O, S. SO, or SO₂, wherein said ring is optionally fused to a (C6-C10)aryl, (C5-C10)heteroaryl, (C3-C10)cycloalkyl, or a (C3-C10)heterocyclyl; and wherein each R⁵ group is independently and optionally substituted with one or more R⁷;
R⁶ is selected from: —R⁵, —C(O)R⁵, —C(O)OR⁵, —C(O)N(R⁵)₂ and —S(O)₂R⁵;
each occurrence of R⁷ is independently selected from halogen, —OR⁸, —NO₂, —CN, —CF₃, —OCF₃, —R⁸, oxo, thioxo, 1,2-methylenedioxy, 1,2-ethylenedioxy. —N(R⁸)₂, —SR⁸, —SOR⁸, —SO₂R⁸, —SO₂N(R⁸)₂, —SO₃R⁸, —C(O)R⁸, —C(O)C(O)R⁸, —C(O)CH2C(O)R⁸, —C(S)R⁸, —C(S)0R⁸, —C(O)OR⁸, —C(O)C(O)OR⁸, —C(O)C(O)N(R⁸)₂, —OC(O)R⁸, —C(O)N(R⁸)₂, —OC(O)N(R⁸)₂, —C(S)N(R⁸)₂, —(CH₂)_{0—2}NHC(O)R⁸, —N(R⁸)N(R⁸)COR⁸, —N(R⁸)N(R⁸)C(O)OR⁸, —N(R⁸)N(R⁸)CON(R⁸)₂, —N(R⁸)SO₂R⁸, —N(R⁸)SO₂N(R⁸)₂, —N(R⁸)C(O)OR⁸, —N(R⁸)C(O)R⁸, —N(R⁸)C(S)R⁸, —N(R⁸)C(O)N(R⁸)₂, —N(R⁸)C(S)N(R⁸)₂, —N(COR⁸)COR⁸, —N(OR⁸)R⁸, —C(═NH)N(R⁸)₂, —C(O)N(OR⁸)R⁸, —C(═NOR⁸)R⁸, —OP(O)(OR⁸)₂, —P(O)(R⁸)₂, —P(O)(OR⁸)₂, or —P(O)(H)(OR⁸); and
each occurrence of R⁸ is independently selected from: H— and (C1-C6)-aliphatic-.

6. The method of claim 4, wherein the uridine diphosphate derivative is a compound of formula II or a salt or prodrug thereof: 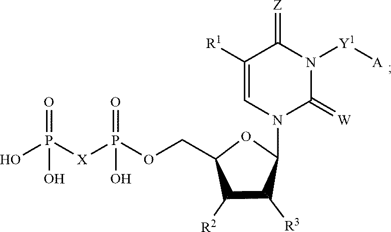
or a prodrug or salt thereof, wherein:
A is selected from: a phenyl group that is substituted with at least one (C1-C5)-aliphatic group or halogen: a naphthalene group; a 5- to 10-membered heteroaryl group having up to 5 heteroatoms independently selected from N, O and S; and a 3- to 10-membered non-aromatic ring having up to 5 heteroatoms independently selected from N, O, S. SO, or SO₂; wherein A is optionally further substituted with one or more R⁴;
X is independently selected from —O—, —S—, —N(R⁵)— and a (C1-C3)-aliphatic group independently and optionally substituted with one or more R⁴;
Y¹ is a (C1-C5)-aliphatic group substituted with at least one oxo and further independently and optionally substituted with one or more R⁴;
Z and W are each independently selected from ═O, ═S, ═N(R⁵), and ═NOR⁵;
R¹ is selected from: —H, halogen, —OR⁵, —CN, —CF₃, —OCF₃ and a (C1-C6)-aliphatic-group optionally substituted with one or more R⁴;
R² and R³ are each independently selected from —OR⁵, —SR⁵, —NR⁵R⁶, —OC(O)R⁵, —OC(O)NR⁵R⁶, and —OC(O)OR⁵; preferably, R² and R³ are each independently selected from —OR⁵, —SR⁵, —NR⁵R⁶ and —OC(O)R⁵;
each occurrence of R⁴ is independently selected from: halogen, —OR⁵, —NO₂, —CN, —CF₃, —OCF₃, —R⁵, oxo, thioxo, 1,2-methylenedioxy, 1,2-ethylenedioxy, —N(R⁵)₂, —SR⁵, —SOR⁵, —SO₂R⁵, —SO₂N(R⁵)₂, —SO₃R⁵, —C(O)R⁵, —C(O)C(O)R⁵, —C(O)CH₂C(O)R⁵, —C(S)R⁵, —C(S)OR⁵, —C(O)OR⁵, —C(O)C(O)OR⁵, —C(O)C(O)N(R⁵)₂, —OC(O)R⁵, —C(O)N(R⁵)₂, —OC(O)N(R⁵)₂, —C(S)N(R⁵)₂, —(CH₂)_{0—2}NHC(O)R⁵, —N(R⁵)N(R⁵)COR⁵, —N(R⁵)N(R⁵)C(O)OR⁵, —N(R⁵)N(R⁵)CON(R⁵)₂, —N(R⁵)SO₂R⁵, —N(R⁵)SO₂N(R⁵)₂, —N(R⁵)C(O)OR⁵, —N(R⁵)C(O)R⁵, —N(R⁵)C(S)R⁵, —N(R⁵)C(O)N(R⁵)₂, —N(R⁵)C(S)N(R⁵)₂, —N(COR⁵)COR⁵, —N(OR⁵)R⁵, —C(═NH)N(R⁵)₂, —C(O)N(OR⁵)R⁵, —C(═NOR⁵)R⁵, —OP(O)(OR⁵)₂, —P(O)(R⁵)₂, —P(O)(OR⁵)₂, or —P(O)(H)(OR⁵);
each occurrence of R⁵ is independently selected from H—, (C1-C12)-aliphatic-, (C3-C10)-cycloalkyl- or -cycloalkenyl-, [(C3-C10)-cycloalkyl or -cycloalkenyl]-(C1-C12)-aliphatic-, (C6-C10)-aryl-, (C6-C10)-aryl-(C1-C12)aliphatic-, (C3-C10)-heterocyclyl-, (C6-C10)-heterocyclyl-(C1-C12)aliphatic-, (C5-C10)-heteroaryl-, and (C5-C10)-heteroaryl-(C1-C12)-aliphatic-; wherein two R⁵ groups bound to the same atom optionally form a 3- to 10-membered aromatic or non-aromatic ring having up to 3 heteroatoms independently selected from N, O, S, SO. or SO₂, wherein said ring is optionally fused to a (C6-C10)aryl, (C5-C10)heteroaryl, (C3-C10)cycloalkyl, or a (C3-C10)heterocyclyl; and wherein each R⁵ group is independently and optionally substituted with one or more R⁷;
R⁶ is selected from: R⁵, —C(O)R⁵, —C(O)OR⁵, —C(O)N(R⁵)₂ and —S(O)₂R⁵;
each occurrence of R⁷ is independently selected from: halogen. —OR⁸, —NO₂, —CN, —CF₃, —OCF₃, —R⁸, oxo, thioxo, 1,2-methylenedioxy, 1,2-ethylenedioxy. —N(R⁸)₂, —SR⁸, —SOR^{B}, —SO₂R⁸, —SO₂N(R⁸)₂, —SO₃R⁸, —C(O)R⁸, —C(O)C(O)R⁸, —C(O)CH2C(O)R⁸, —C(S)R⁸, —C(S)OR⁸, —C(O)OR⁸, —C(O)C(O)OR⁸, —C(O)C(O)N(R⁸)₂, —OC(O)R⁸, —C(O)N(R⁸)₂, —OC(O)N(R⁸)₂, —C(S)N(R⁸)₂, —(CH₂)_{0—2}NHC(O)R⁸, —N(R⁸)N(R⁸)COR⁸, —N(R⁸)N(R⁸)C(O)OR⁸, —N(R⁸)N(R⁸)CON(R⁸)₂, —N(R⁸)SO₂R⁸, —N(R⁸)SO₂N(R⁸)₂, —N(R⁸)C(O)OR⁸, —N(R⁸)C(O)R⁸, —N(R⁸)C(S)R⁸, —N(R⁸)C(O)N(R⁸)₂, —N(R⁸)C(S)N(R⁸)₂, —N(COR⁸)COR⁸, —N(OR⁸)R⁸, —C(═NH)N(R⁸)₂, —C(O)N(OR⁸)R⁸, —C(═NOR⁸)R⁸, —OP(O)(OR⁸)₂, —P(O)(R⁸)₂, —P(O)(OR⁸)₂, or —P(O)(H)(OR⁸)— and
each occurrence of R⁸ is independently selected from: H— and (C1-C6)-aliphatic-.

7. The method of any one of claims 1 to 6, wherein the P₂Y receptor modulating compound is administered topically, orally, or intravenously.

8. The method of any one of claims 1 to 7, wherein the P₂Y receptor modulating compound is administered ophthalmically.

9. The method of any one claims 1 to 8, wherein the P₂Y receptor modulating compound is formulated in an ophthalmically acceptable carrier.

10. The method of claim 9, wherein the ophthalmically acceptable carrier comprises one or more of an effective amount of a buffer component; an effective amount of a tonicity component; an effective amount of a preservative component; and water.

11. The method of any one of claims 1 to 10, wherein the P₂Y receptor modulating compound is administered in combination with a second therapeutic agent.

12. The method of claim 11, wherein the second therapeutic agent is selected from the group consisting of: NMDA antagonists, antibacterials, antihistamines, decongestants, antiinflammatories, antiparasitics, miotics, sympathomimetics, anticholinergics, adrenergics, antivirals, secretagogues exemplified by agents that promote lacrimation, salivation or stimulation of release of soluble mucins and or expression of cell associated mucins that promote wettability and/or lubricity of mucosal surfaces, local anesthetics, antifungals, amoebicidals, trichomonocidals, analgesics, mydriatics, antiglaucoma drugs, carbonic anhydrase inhibitors, ophthalmic diagnostic agents, ophthalmic agents used as adjuvants in surgery, chelating agents, antineoplastics, antihypertensives, muscle relaxants, diagnostics, adrenergic anesthetics, beta blockers, alpha-2-agonists, cycloplegics, prostaglandins, derivatives thereof and mixtures thereof.

13. A P₂Y receptor modulating compound for use in treating an eye disorder in a subject in need thereof.

14. Use of claim 13, wherein the eye disorder is selected from the group consisting of retinal neurodegenerations, including glaucoma, age-related macular degeneration and traumatic retinal injury, posterior and anterior uveitis of the eye, and dry eye disorders.

15. Use of any of claims 13 to 14, wherein the P₂Y receptor modulating compound is a P₂Y₆ receptor modulating compound.

16. Use of any one of claims 13 to 15, wherein the P2Y receptor modulating compound is a uridine diphosphate derivative or prodrug thereof.

17. Use of claim 16, wherein the uridine diphosphate derivative is a compound of formula I or a salt or a prodrug thereof: 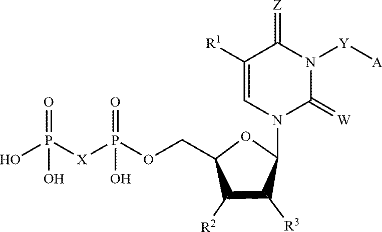
or a salt thereof, wherein:
A is 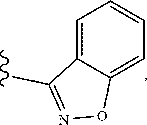
wherein A is optionally substituted with one or more R⁷;
X is independently selected from —O—, —S—, —N(R⁵)— and a (C1-C3)-aliphatic group independently and optionally substituted with one or more R⁴;
Y is a bond or a (C1-C5)-aliphatic group independently and optionally substituted with one or more R⁴;
Z and W are each independently selected from =O, =S. ═N(R⁵), and ═NOR⁵;
R¹ is selected from: —H, halogen. —OR⁵, —CN, —CF₃, —OCF₃ and a (C1-C6)-aliphatic group optionally substituted with one or more R⁷;
R² and R³ are each independently selected from —OR⁵, —SR⁵, —NR⁵R⁶, —OC(O)R⁵, —OC(O)NR⁵R⁶, and —OC(O)OR⁵;
each occurrence of R⁴ is independently selected from halogen, —OR⁵, —NO₂, —CN, —CF₃, —OCF₃, —R⁵, 1,2-methylenedioxy, 1,2-ethylenedioxy, —N(R⁵)₂, —SR⁵, —SOR⁵, —SO₂R⁵, —SO₂N(R⁵)₂, —SO₃R⁵, —C(O)R⁵, —C(O)C(O)R⁵, —C(O)CH₂C(O)R⁵, —C(S)R⁵, —C(S)OR⁵, —C(O)OR⁵, —C(O)C(O)OR⁵, —C(O)C(O)N(R⁵)₂, —OC(O)R⁵, —C(O)N(R⁵)₂, —OC(O)N(R⁵)₂, —C(S)N(R⁵)₂, —(CH₂)_{0—2}NHC(O)R⁵, —N(R⁵)N(R⁵)COR⁵, —N(R⁵)N(R⁵)C(O)OR⁵, —N(R⁵)N(R⁵)CON(R⁵)₂, —N(R⁵)SO₂R⁵, —N(R⁵)SO₂N(R⁵)₂, —N(R⁵)C(O)OR⁵, —N(R⁵)C(O)R⁵, —N(R⁵)C(S)R⁵, —N(R⁵)C(O)N(R⁵)₂, —N(R⁵)C(S)N(R⁵)₂, —N(COR⁵)COR⁵, —N(OR⁵)R⁵, —C(═NH)N(R⁵)₂, —C(O)N(OR⁵)R⁵, —C(═NOR⁵)R⁵, —OP(O)(OR⁵)₂, —P(O)(R⁵)₂, —P(O)(OR⁵)₂, or —P(O)(H)(OR⁵);
each occurrence of R⁵ is independently selected from: H—, (C1-C12)-aliphatic-. (C3-C 10)-cycloalkyl- or -cycloalkenyl-. [(C3-C10)-cycloalkyl or -cycloalkenyl-]-(C1-C12)-aliphatic-, (C6-C10)-aryl-, (C6-C10)-aryl-(C1-C12)aliphatic-, (C3-C10)-heterocyclyl-, (C6-C10)-heterocyclyl-(C1-C12)aliphatic-, (C5-C10)-heteroaryl-, and (C5-C10)-heteroaryl-(C1-C12)-aliphatic-; wherein two R⁵ groups bound to the same atom optionally form a 3- to 10-membered aromatic or non-aromatic ring having up to 3 heteroatoms independently selected from N, O, S. SO, or SO₂, wherein said ring is optionally fused to a (C6-C10)aryl, (C5-C10)heteroaryl, (C3-C10)cycloalkyl, or a (C3-C10)heterocyclyl; and wherein each R⁵ group is independently and optionally substituted with one or more R⁷;
R⁶ is selected from: —R⁵, —C(O)R⁵, —C(O)OR⁵, —C(O)N(R⁵)₂ and —S(O)₂R⁵;
each occurrence of R⁷ is independently selected from: halogen, —OR⁸, —NO₂, ——NO₂, —CN, ——CF₃, —OCF₃, —R⁸, oxo, thioxo, 1,2-methylenedioxy, 1,2-ethylenedioxy, —N(R⁸)₂, —SR⁸, — SOR⁸, —SO₂R⁸, —SO₂N(R⁸)₂, —SO₃R⁸, —C(O)R⁸, —C(O)C(O)R⁸, —C(O)CH₂C(O)R⁸, —C(S)R⁸, —C(S)0R⁸, —C(O)OR⁸, —C(O)C(O)OR⁸, —C(O)C(O)N(R⁸)₂,—OC(O)R⁸,—C(O)N(R⁸)₂, —OC(O)N(R⁸)₂, —C(S)N(R⁸)₂, —(CH₂)_{0—2}NHC(O)R⁸, —N(R⁸)N(R⁸)COR⁸, — N(R⁸)N(R⁸)C(O)OR⁸, —N(R⁸)N(R⁸)CON(R⁸)₂, —N(R⁸)SO₂R⁸, —N(R⁸)SO₂N(R⁸)₂, — N(R⁸)C(O)OR⁸, —N(R⁸)C(O)R⁸, —N(R⁸)C(S)R⁸, —N(R⁸)C(O)N(R⁸)₂, —N(R⁸)C(S)N(R⁸)₂, —N(COR⁸)COR⁸, —N(OR⁸)R⁸, —C(═NH)N(R⁸)₂, —C(O)N(OR⁸)R⁸, —C(═NOR⁸)R⁸,—OP(O)(OR⁸)₂, —P(O)(R⁸)₂, —P(O)(OR⁸)₂, or —P(O)(H)(OR⁸); and
each occurrence of R⁸ is independently selected from: H— and (C1-C6)-alipliatic-.

18. Use of claim 15, wherein the uridine diphosphate derivative is a compound of formula II or a salt or prodrug thereof: 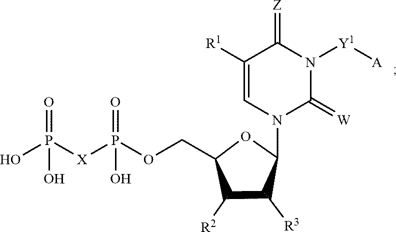
or a prodrug or salt thereof, wherein:
A is selected from: a phenyl group that is substituted with at least one (C1-C5)-aliphatic group or halogen: a naphthalene group; a 5- to 10-membered heleroaryl group having up to 5 heteroatoms independently selected from N, O and S; and a 3- to 10-membered non-aromatic ring having up to 5 heteroatoms independently selected from N, O, S, SO, or SO₂; wherein A is optionally further substituted with one or more R⁴:
X is independently selected from —O—, —S—, —N(R⁵)— and a (C1-C3)-aliphatic: group independently and optionally substituted with one or more R⁴;
Y¹ is a (C1-C5)-aliphatic group substituted with at least one oxo and further independently and optionally substituted with one or more R^{4;}
Z and W are each independently selected from: ═O. ═S, ═N(R⁵), and ═NOR⁵;
R¹ is selected from: —H, halogen, —OR⁵, —CN, —CF₃, —OCF₃ and a (C1-C6)-ahphaticgroup optionally substituted with one or more R⁴—
R² and R³ are each independently selected from —OR⁵, —SR⁵, —NR⁵R⁶, —OC(O)R⁵, —OC(O)NR⁵R⁶, and —OC(O)OR⁵; preferably, R² and R³ are each independently selected from —OR⁵, —SR⁵, —NR⁵R⁶ and —OC(O)R⁵;
each occurrence of R⁴ is independently selected from: halogen, —OR⁵, —NO₂, —CN, —CF₃, —OCF₃,—R⁵, oxo, thioxo, 1,2-methylenedioxy, 1,2-ethylenedioxy, —N(R⁵)₂, —SR⁵, — SOR⁵, —SO₂R⁵, —SO₂N(R⁵)2, —SO₃—R⁵, —C(O)R⁵, —C(O)C(O)R5, —C(O)CH2C(O)R⁵, ——C(S)R⁵, —C(S)OR⁵, —C(O)OR⁵, —C(O)C(O)OR⁵, —C(O)C(O)N(R⁵)₂, —OC(O)R⁵, —C(O)N(R⁵)₂, —OC(O)N(R⁵)₂, —C(S)N(R⁵)₂, —(CH₂)₀—₂NHC(O)R⁵, —N(R⁵)N(R⁵)COR⁵, — N(R⁵)N(R⁵)C(O)OR⁵, —N(R⁵)N(R⁵)CON(R⁵)₂,—N(R⁵)SO₂R5, —N(R⁵)SO2N(R⁵)₂, —N(R⁵)(C(O)OR⁵, —N(R⁵)C(O)R⁵, —N(R⁵)C(S)R⁵, —N(R⁵)C(O)N(R⁵)₂, —N(R⁵)C(S)N(R⁵)₂, —N(COR⁵)COR⁵, —N(OR⁵)R⁵, —C(═N—H)N(R⁵)₂, —C(O)N(OR⁵)R⁵, —C(═NOR⁵)R⁵, — OP(O)(OR⁵)₂, —P(O)(R⁵)₂, —P(O)(OR⁵)₂, or —P(O)(H)(OR⁵);
each occurrence of R⁵ is independently selected from: H—, (C1-C12)-aliphatic-, (C3-C10)-cycloalkyl- or -cycloalkenyl-, [(C3-C10)-cycloalkyl or -cycloalkenyl[-(C1-C12)-aliphatic-, (C6-C10)-aryl-, (C6-C10)-aryl-(C1-C12)aliphatic-,(C3-C10)-heterocyclyl-, (C6-C10)-heterocyciyl-(C1-C12)aliphatic:-, (C5-C10)-heteroaryl-, and (C5-C10-heteroary-1-(C1-C12)-aliphatic-: wherein two R⁵ groups bound to the same atom optionally form a 3- to 10-membered aromatic or non-aromatic ring having up to 3 heteroatoms independently selected from N, O, S, SO. or SO₂, wherein said ring is optionally fused to a (C6-C10)aryl, (C5-C10)heteroaryl, (C3-C10)cyeloalkyl, or a (C3-C 10)heterocyclyl; and wherein each R⁵ group is independently and optionally substituted with one or more R⁷;
R⁶ is selected from: R⁵, —C(O)R⁵, —C(O)OR⁵, —C(O)N(R⁵)₂ and —S(O)₂R⁵;
each occurrence of R⁷ is independently selected from: halogen, —OR⁸, —NO₂, —CN, —CF₃, —OCF₃, —R⁸, oxo, thioxo, 1,2-methylenedioxy, 1,2-ethylenedioxy, —N(R⁸)₂, —SR⁸, —SOR^{B}, —SO₂R⁸, —SO₂N(R⁸)₂, —SO₃R⁸, —C(O)R⁸, —C(O)C(O)R⁸, —C(O)CH₂(O)R⁸, —C′(S)R⁸, —C(S)OR⁸, —C(O)OR⁸, —C(O)C(O)OR⁸, —C(O)C(O)N(R⁸)₂, —OC(O)R⁸, — C(O)N(R⁸)₂, —OC(O)N(R⁸)₂, —C(S)N(R⁸)₂, —(CH₂)O₂NHC(O)R⁸, —N(R⁸)N(R⁸)COR⁸, —N(R⁸)N(R⁸)C(O)OR⁸, —N(R⁸)N(R⁸)CON(R⁸)₂, —N(R⁸)N(R⁸)CON(R⁸)₂, —N(R⁸)SO₂R⁸,—N(R⁸)C(O)OR⁸, —N(R⁸)C(O)R⁸, —N(R⁸)C(S)R⁸, —N(R⁸)C(O)N(R⁸)₂, —N(R⁸)C(S)N(R⁸)₂, —N(COR⁸)COR⁸, —N(OR⁸)R⁸, —C(═NH)N(R⁸)₂, —C(O)N(OR⁸)R⁸, —C(═NOR⁸)R⁸, —OP(O)(OR⁸)₂, —P(O)(R⁸)₂, —P(O)(OR⁸)₂, or —P(O)(H)(OR⁸); and
each occurrence of R⁸ is independently selected from: H— and (C1-C6)-aliphatic-.

19. Use of any one of claims 13 to 18, wherein the P₂Y receptor modulating compound is administered topically, orally, or intravenously.

20. Use of any one of claims 13 to 19, wherein the P₂Y receptor modulating compound is administered ophthalmically.

21. Use of any one of claims 13 to 20, wherein the P₂Y receptor modulating compound is formulated in an ophthalmically acceptable carrier.

22. Use of claim 21, wherein the ophthalmically acceptable carrier comprises one or more of an effective amount of a buffer component; an effective amount of a tonicity component; an effective amount of a preservative component; and water.

23. Use of any one of claims 13 to 22 wherein the P₂Y receptor modulating compound is delivered via a route selected from the group consisting of topical, subconjunctival, intracameral, intravitreal, suprachoroidal, subretinal and retrobulbar routes.

24. Use of any one of claims 13 to 23, wherein the P₂Y receptor modulating compound is formulated in a controlled release formulation.

25. Use of claim 24, wherein the controlled release formulation comprises an agent selected from the group consisting of a solid polymer formulation and a hydrogel formulation.

26. Use of any one of claims 13 to 25, wherein the P₂Y receptor modulating compound is administered in combination with a second therapeutic agent.

27. Use of claim 26, wherein the second therapeutic agent is selected from the group consisting of: NMDA antagonists, antibacterials, antihistamines, decongestants, antiinflammatories, antiparasitics, miotics, sympathomimetics, anticholinergics, adrenergics, antivirals, secretagogues exemplified by agents that promote lacrimation, salivation or stimulation of release of soluble mucins and or expression of cell associated mucins that promote wettability and/or lubricity of mucosal surfaces, local anesthetics, antifungals, amoebicidals, trichomonocidals, analgesics, mydriatics, antiglaucoma drugs, carbonic anhydrase inhibitors, ophthalmic diagnostic agents, ophthalmic agents used as adjuvants in surgery, chelating agents, antineoplastics, antihypertensives, muscle relaxants, diagnostics, adrenergic anesthetics, beta blockers, alpha-2-agonists, cycloplegics, prostaglandins, derivatives thereof and mixtures thereof.

28. An ophthalmically acceptable formulation for administration to the eye comprising an effective amount of a P₂Y receptor modulating compound and one or more of an effective amount of a buffer component; an effective amount of a tonicity component; an effective amount of a preservative component; and water.

29. Formulation of claim 28, wherein the effective amount a P₂Y receptor modulating compound is a dosage sufficient to treat an eye disorder selected from the group consisting of retinal neurodegenerations, including glaucoma, age-related macular degeneration and traumatic retinal injury, posterior and anterior uveitis of the eye, and dry eye disorders, when administered one or more times daily or delivered in a long acting controlled release formulation.

30. Formulation of any of claims 28 to 29, wherein the P₂Y receptor modulating compound is a P₂Y₆ receptor modulating compound.

31. Formulation of any one of claims 28 to 30, wherein the P2Y receptor modulating compound is a uridine diphosphate derivative or prodrug thereof.

32. Formulation of claim 31, wherein the uridine diphosphate derivative is a compound of formula I or a salt or a prodrug thereof. 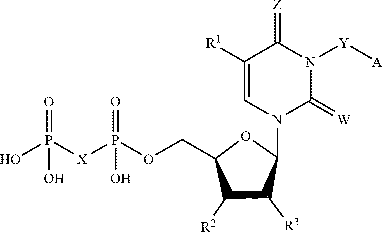
or a salt thereof, wherein.
A is 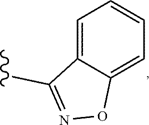
wherein A is optionally substituted with one or more R⁷:
X is independently selected from —O—, —S—, —N(R⁵)— and a (C l-C3)-aliphatic group independently and optionally substituted with one or more R⁴:
Y is a bond or a (C1-C5)-aliphatic group independently and optionally substituted with one or more R⁴:
Z and W are each independently selected from ═O, ═S, ═N(R⁵), and ═NOR⁵;
R¹ is selected from: —H, halogen, —OR⁵, —CN, —CF₃, —OCF₃ and a (Cl-C6)-aliphatic group optionally substituted with one or more R⁷,
R² and R³ are each independently selected from —OR⁵, —SR⁵, —NR⁵R⁶, —OC(O)R⁵, —OC(O)NR⁵R⁶, and —OC(O)OR⁵;
each occurrence of R⁴ is independently selected from: halogen. —OR⁵, —NO₂, —CN, —CF₃, —OCF₃, —R⁵, 1,2-methylenedioxy. 1,2-ethylenedioxy, —N(R⁵)₂, —SR⁵, —SOR⁵, —SO₂R⁵, —SO₂N(R⁵)₂, —SO₃R⁵, —C(O)R⁵, —C(O)C(O)R⁵, —C(O)CH₂C(O)R⁵, —C(S)R⁵, —C(S)OR⁵, —C(O)OR⁵, —C(O)C(O)OR⁵, —C(O)C(O)N(R⁵)₂, —OC(O)R⁵, —C(O)N(R⁵)₂, — OC(O)N(R⁵)₂, —C(S)N(R⁵)₂, —(CH₂)_{0—2}NHC(O)R⁵, —N(R⁵)N(R⁵)COR⁵, —N(R⁵)N(R⁵)C(O)OR⁵, —N(R⁵)N(R⁵)CON(R⁵)₂, —N(R⁵)SO₂R⁵, —N(R⁵)SO2N(R⁵)₂, —N(R⁵)C(O)OR⁵, —N(R⁵)C(O)R⁵, —N(R⁵)C(S)R⁵, —N(R⁵)C(O)N(R⁵)₂, —N(R⁵)C(S)N(R⁵)₂, —N(COR⁵)COR⁵, —N(OR⁵)R⁵, —C(═NH)N(R⁵)₂, —C(O)N(OR⁵)R⁵, —C(═NOR⁵)R⁵, —OP(O)(OR⁵)2, —P(O)(R5)₂, —P(O)(OR⁵)₂, or —P(O)(H)(OR⁵);
each occurrence of R⁵ is independently selected from: H—, (C1-C12)-aliphatic-, (C3-C10)-cycloalkyl- or -cycloalkenyl-, [(C3-C10)-cycloalkyl or -cxcloalkenyl-]-(Cl-C12)-aliphatic-, (C6-C10)-aryl-. (C6-C10)-aryl-(C1-C12)aliphatic, (C3-C-10)-heterocyclyl-, C(C6-C10)-heterocyclyl-(C1-C12)aliphatic-, (C5-C10)-heteroaryl-, and (C5-C10)-heteroaryl-(C1-C12)-aliphatic-: wherein two R⁵ groups bound to the same atom optionally form a 3- to 10-membered aromatic or non-aromatic ring having up to 3 heteroatoms independently selected from N, O, S, SO. or SO₂, wherein said ring is optionally fused to a (C6-C10)aryl, (C5-C10)heteroaryl, (C3-C10)cyeloalkyl, or a (C3-C10)heterocyclyl; and wherein each R⁵ group is independently and optionally substituted with one or more R⁷;
R⁶ is selected from: —R⁵, —C(O)R⁵, —C(O)OR⁵, —C(O)N(R⁵)₂ and —S(O)₂R⁵;
each occurrence of R⁷ is independently selected from: halogen, —OR⁸, —NO₂, —CN, —CF₃, —OCF₃, —R⁸, oxo, thioxo, 1,2-methylenedioxy. 1,2-ethylenedioxy, —N(R⁸)₂, —SR⁸, — SOR⁸, —SO₂R⁸, —SO₂N(R⁸)₂, —SO₃R⁸, —C(O)R⁸, —C(O)C(O)(R⁸, —C(O)CH₂C(O)R⁸_{,} — C(S)R⁸, —C(S)0R⁸, —C(O)OR⁸, —C(O)C(O)OR⁸, —C(O)C(O)N(R⁸)₂, —OC(O)R⁸, —C(O)N(R⁸)₂, —OC(O)N(R⁸)₂, —C(S)N(R⁸)₂, —(CH₂)_{0—2}NHC(O)R⁸, —N(R⁸)N—(R⁸)COR⁸, —N(R⁸)N(R⁸)C(O)OR⁸, —N(R⁸)N(R⁸)CON(R⁸)₂, —N(R⁸)SO₂R⁸, —N(R⁸)SO₂N(R⁸)2, — N(R⁸)C(O)OR8, —N(R⁸)C(O)R⁸, —N(R⁸)C(S)R⁸, —N(R⁸)C(O)N(R⁸)₂, —N(R⁸)C(S)N(R⁸)₂, —N(COR⁸)COR⁸, —N(OR⁸)R⁸, —C(══NH)N(R⁸)₂, —C(O)N(OR⁸)R⁸, —C(══NOR⁸)R⁸, —OP(O)(OR⁸)₂, —P(O)(R⁸)₂, —P(O)(OR⁸)₂, or —P(O)(H)(OR⁸); and
each occurrence of R⁸ is independently selected from: H— and (C1-C6)-aliphatic-.

33. Formulation of claim 28, wherein the uridine diphosphate derivative is a compound of formula II or a salt or prodrug thereof: 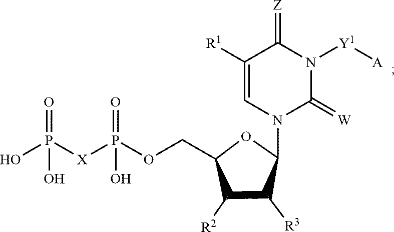
or a prodrug or salt thereof, wherein:
A is selected from: a phenyl group that is substituted with at least one (C1 -C5)-aliphatic group or halogen: a naphthalene group; a 5- to 10-membered heleroaryl group having up to 5 heteroatoms independently selected from N, O and S; and a 3- to 10-membered non-aromatic ring having up to 5 heteroatoms independently selected from N, O, S. SO, or SO₂; wherein A is optionally further substituted with one or more R⁴;
X is independently selected from —O—, —S—, —N(R⁵)— and a (C1-C3)-aliphatic group independently and optionally substituted with one or more R⁴;
Y¹ is a (C1-C5)-aliphatic group substituted with at least one oxo and further independently and optionally substituted with one or more R⁴;
Z and W are each independently selected from ═O, ═S. ═N(R⁵), and ═NOR⁵;
R¹ is selected from: —H, halogen, —OR⁵, —CN, —CF₃, —OCF₃ and a (C1-C6)-aliphatic-group optionally substituted with one or more R⁴;
R² and R³ are each independently selected from —OR⁵, —SR⁵, —NR⁵R⁶, —OC(O)R⁵, — OC(O)NR⁵R⁶, and —OC(O)OR⁵; preferably, R² and R³ are each independently selected from —OR⁵, —SR⁵, —NR⁵R⁶ and —OC(O)R⁵;
each occurrence of R⁴ is independently selected from: halogen, —OR⁵, —NO₂, —CN, —CF₃, —OCF₃, —R⁵, oxo, thioxo, 1,2-methylenedioxy. 1,2-ethylenedioxy, —N(R⁵)₂, —SR⁵, — SOR⁵, —SO₂R⁵, —SO₂N(R⁵)₂, —SO₃R⁵, —C(O)R⁵. —C(O)C(O)R⁵, —C(O)CH₂C(O)R⁵, — C(S)R⁵, —C(S)OR⁵, —C(O)OR⁵, —C(O)C(O)OR⁵, —C(O)C(O)N(R⁵)₂, —OC(O)R⁵, —C(O)N(R⁵)₂, —OC(O)N(R⁵)₂, —C(S)N(R⁵)₂, —(CH₂)_{0—2}NHC(O)R⁵, —N(R⁵)N(R⁵)COR⁵, — N(R⁵)N(R⁵)C(O)OR⁵, —N(R⁵)N(R⁵)CON(R⁵)₂, —N(R⁵)SO₂R⁵, —N(R⁵)SO₂N(R⁵)₂, — N(R⁵)C(O)OR⁵, —N(R⁵)C(O)R⁵, —N(R⁵)C(S)R⁵, —N(R⁵)C(O)N(R⁵)₂, —N(R⁵)C(S)N(R⁵)₂, —N(COR⁵)COR⁵, —N(OR⁵)R⁵, —C(═NH)N(R⁵)₂, —C(O)N(OR⁵)R⁵, —C(═NOR⁵)R⁵, — OP(O)(OR⁵)₂, —P(O)(R⁵)₂, —P(O)(OR⁵)₂, or —P(O)(H)(OR⁵);
each occurrence of R⁵ is independently selected from: H—, (C1-C12)-aliphatic-, (C3-C10)-cycloalkyl- or -cycloalkenyl-, [(C3-C10)-cycloalkyl or -cycloalkenyl]-(C1-C12)-aliphatic-, (C6-C10)-aryl-, (C6-C10)-aryl-(C1-C12)aliphatic-. (C3-C10)-heterocyclyl-, (C6-C10)-heterocyclyl-(C1-C12)aliphatic-, (C5-C10)-heteroaryl-, and (C5-C10)-heteroaryl-(C1-C12)-aliphatic-; wherein two R⁵ groups bound to the same atom optionally form a 3- to 10-membered aromatic or non-aromatic ring having up to 3 heteroatoms independently selected from N, O, S. SO, or SO₂, wherein said ring is optionally fused to a (C6-C10)aryl, (C5-C10)heteroaryl, (C3-C10)cycloalkyl, or a (C3-C10)heterocyclyl; and wherein each R⁵ group is independently and optionally substituted with one or more R⁷—
R⁶ is selected from: R⁵, —C(O)R⁵, —C(O)OR⁵, —C(O)N(R⁵)₂ and —S(O)₂R⁵;
each occurrence of R⁷ is independently selected from: halogen, —OR⁸, —NO₂, —CN, —CF₃, —OCF₃, —R⁸, oxo, thioxo, 1,2-methylenedioxy. 1,2-ethylenedioxy, —N(R⁸)₂, —SR⁸, —SOR^{B}, —SO₂R⁸, —SO₂N(R⁸)₂, —SO₃R⁸, —C(O)R⁸, —C(O)C(O)R⁸, —C(O)CH₂C(O)R⁸, —C(S)R⁸, —C(S)OR⁸, —C(O)OR⁸, —C(O)C(O)OR⁸, —C(O)C(O)N(R⁸)₂, —OC(O)R⁸, —C(O)N(R⁸)₂, —OC(O)N(R⁸)₂, —C(S)N(R⁸)₂, —(CH₂)_{0—2}NHC(O)R⁸, —N(R⁸)N(R⁸)COR⁸, — N(R⁸)N(R⁸)C(O)OR⁸, —N(R⁸)N(R⁸)CON(R⁸)₂, —N(R⁸)SO₂R⁸, —N(R⁸)SO₂N(R⁸)₂, — N(R⁸)C(O)OR⁸, —N(R⁸)C(O)R⁸, —N(R⁸)C(S)R⁸, —N(R⁸)C(O)N(R⁸)₂, —N(R⁸)C(S)N(R⁸)₂, —N(COR⁸)COR⁸, —N(OR⁸)R⁸, —C(═NH)N(R⁸)₂, —C(O)N(OR⁸)R⁸, —C(═NOR⁸)R⁸, —OP(O)(OR⁸)₂, —P(O)(R⁸)₂, —P(O)(OR⁸)₂, or —P(O)(H)(OR⁸); and
each occurrence of R⁸ is independently selected from: H— and (C1-C6)-aliphatic-.

34. Formulation of any one of claims 28 to 33, wherein the formulation comprises a second therapeutic agent.

35. Formulation of claim 34, wherein the second therapeutic agent is selected from the group consisting of: NMDA antagonists, antibacterials, antihistamines, decongestants, antiinflammatories, antiparasitics, miotics, sympathomimetics, anticholinergics, adrenergics, antivirals, local anesthetics, secretagogues exemplified by but not limited to agents that promote lacrimation, salivation or stimulation of release of soluble mucins and or expression of cell associated mucins that promote wettability and/or lubricity of mucosal surfaces including the ocular surface), antifungals, amoebicidals, trichomonocidals, analgesics, mydriatics, antiglaucoma drugs, carbonic anhydrase inhibitors, ophthalmic diagnostic agents, ophthalmic agents used as adjuvants in surgery, chelating agents, antineoplastics, antihypertensives, muscle relaxants, diagnostics, adrenergic anesthetics, beta blockers, alpha-2-agonists, cycloplegics, prostaglandins, derivatives thereof and mixtures thereof.

36. Formulation of any one of claims 28 to 35 wherein the P₂Y receptor modulating compound is a formulation selected from the group consisting of topical, subconjunctival, intracameral, intravitreal, suprachoroidal, subretinal and retrobulbar formulations.

37. Formulation of any one of claims 28 to 35, wherein the P₂Y receptor modulating compound is formulated in a controlled release formulation.

38. Use of claim 37, wherein the controlled release formulation comprises an agent selected from the group consisting of a solid polymer formulation and a hydrogel formulation.
